SEQUENCE LISTING (37 sequences)

SEQ ID NO: 1 - protein, 16 aa, synthetic construct
RSSQSLVYSNGNTHLN

SEQ ID NO: 2 - protein, 7 aa, synthetic construct
LVSNRDS

SEQ ID NO: 3 - protein, 7 aa, synthetic construct
LVSVRDS

SEQ ID NO: 4 - protein, 9 aa, synthetic construct
VQGTHFPYT

SEQ ID NO: 5 - protein, 6 aa, synthetic construct
NSYWLE

SEQ ID NO: 6 - protein, 5 aa, synthetic construct
SYWLE

SEQ ID NO: 7 - protein, 6 aa, synthetic construct
IRYWLE

SEQ ID NO: 8 - protein, 17 aa, synthetic construct
EILPGIGSTNYNQKFKG

SEQ ID NO: 9 - protein, 17 aa, synthetic construct
EILPGSGSTNYNQKFKG

SEQ ID NO: 10 - protein, 17 aa, synthetic construct
EILPGSGGTIYNQKFKG

SEQ ID NO: 11 - protein, 17 aa, synthetic construct
EILPGTGSTSYNQKFKG

SEQ ID NO: 12 - protein, 17 aa, synthetic construct
EILPGSGGTNYNQKFKG

SEQ ID NO: 13 - protein, 8 aa, synthetic construct
EGWYFDVW

SEQ ID NO: 14 - protein, 112 aa, synthetic construct
DVVMTQSPLSLPVTLGQPASISCRSSQSLVYSNGNTHLNWLQQRPGQSPRRLIYLVSNRDSGVPDRFSGSGSGTDFTLKISRVEAEDVGVYYCVQGTHFPYTFGQGTKLEIK

SEQ ID NO: 15 - protein, 112 aa, synthetic construct
DVVMTQSPLSLPVTLGQPASISCRSSQSLVYSNGNTHLNWLQQRPGQSPRRLIYLVSVRDSGVPDRFSGSGSGTDFTLKISRVEAEDVGVYYCVQGTHFPYTFGQGTKLEIK

SEQ ID NO: 16 - protein, 116 aa, synthetic construct
QVQLQQSGAEVMKPGASVKVSCKASGYTLNSYWLEWVRQRPGHGLEWIGEILPGIGSTNYNQKFKGRVTFTADTSTNTAYMELSSLTSEDTAVYYCAREGWYFDVWGAGTTVTVSS

SEQ ID NO: 17 - protein, 116 aa, synthetic construct
QVQLQQSGAEVMKPGASVKVSCKASGYTLSSYWLEWVRQRPGHGLEWIGEILPGSGSTNYNQKFKGRVTFTADTSTNTAYMELSSLTSEDTAVYYCAREGWYFDVWGAGTTVTVSS

SEQ ID NO: 18 - protein, 116 aa, synthetic construct
QVQLQQSGAEVMKPGASVKVSCKASGYTLNSYWLEWVRQRPGHGLEWIGEILPGSGGTIYNQKFKGRVTFTADTSTNTAYMELSSLTSEDTAVYYCAREGWYFDVWGAGTTVTVSS

SEQ ID NO: 19 - protein, 116 aa, synthetic construct
QVQLQQSGAEVMKPGASVKVSCKASGYTLNSYWLEWVRQRPGHGLEWIGEILPGTGSTSYNQKFKGRVTFTADTSTNTAYMELSSLTSEDTAVYYCAREGWYFDVWGAGTTVTVSS

SEQ ID NO: 20 - protein, 116 aa, synthetic construct
QVQLQQSGAEVMKPGASVKVSCKASGYTLIRYWLEWVRQRPGHGLEWIGEILPGSGGTNYNQKFKGRVTFTADTSTNTAYMELSSLTSEDTAVYYCAREGWYFDVWGAGTTVTVSS

SEQ ID NO: 21 - DNA, 336 bp, synthetic construct
  source 1..336: mol_type = other DNA
gatgttgtgatgactcagtctcctctgagccttcctgttactcttggtcagcctgctagcatctcttgcaggtcatctcagtctctggtgtacagcaatggtaacacccaccttaactggcttcaacagaggcctggtcaatctcctagaaggcttatctacctggtgagcaacagagactctggtgtgcctgataggttcagcggttctggttctggtactgacttcaccctgaagatcagcagagttgaggctgaggatgtgggagtttactactgcgttcagggtactcacttcccttacactttcggtcagggcaccaagcttgagattaag

SEQ ID NO: 22 - DNA, 336 bp, synthetic construct
  source 1..336: mol_type = other DNA
gatgttgtgatgactcagtctcctctgagccttcctgttactcttggtcagcctgctagcatctcttgcaggtcatctcagtctctggtgtacagcaatggtaacacccaccttaactggcttcaacagaggcctggtcaatctcctagaaggcttatctacctggtgagcgtgagagattctggtgtgcctgataggttcagcggttctggttctggtactgacttcaccctgaagatcagcagagttgaggctgaggatgtgggagtttactactgcgttcagggtactcacttcccttacactttcggtcagggcaccaagcttgagattaag

SEQ ID NO: 23 - DNA, 348 bp, synthetic construct
  source 1..348: mol_type = other DNA
caggttcaacttcaacagtcaggcgctgaggttatgaagcctggtgcttctgtgaaagtgagctgcaaggcttctggttacaccctgaactcttactggcttgagtgggttagacagaggcctggtcatggtcttgaatggattggtgagatcctgcctggtatcggcagcactaattacaaccagaagttcaagggccgagtcaccttcactgctgatacttctactaacaccgcctacatggaactgtccagccttacttctgaggacaccgctgtttactactgcgctagagaaggctggtacttcgatgtttggggtgctggtactaccgtgaccgtttcttct

SEQ ID NO: 24 - DNA, 348 bp, synthetic construct
  source 1..348: mol_type = other DNA
caggttcaacttcaacagtcaggcgctgaggttatgaagcctggtgcttctgtgaaagtgagctgcaaggctagcggttacaccctgtcatcttattggcttgagtgggtgagacagaggcctggtcatggtcttgaatggattggtgagatcctgcctggtagcggtagcactaattacaaccagaagttcaagggccgagtcaccttcactgctgatacttctactaacaccgcctacatggaactgtccagccttacttctgaggacaccgctgtttactactgcgctagagaaggctggtacttcgatgtttggggtgctggtactaccgtgaccgtttcttct

SEQ ID NO: 25 - DNA, 348 bp, synthetic construct
  source 1..348: mol_type = other DNA
caggttcaacttcaacagtcaggcgctgaggttatgaagcctggtgcttctgtgaaagtgagctgcaaggcttctggttacaccctgaactcttactggcttgagtgggttagacagaggcctggtcatggtcttgaatggattggtgagatcctgcctggttctggtggcactatctacaaccagaagttcaagggccgagtcaccttcactgctgatacttctactaacaccgcctacatggaactgtccagccttacttctgaggacaccgctgtttactactgcgctagagaaggctggtacttcgatgtttggggtgctggtactaccgtgaccgtttcttct

SEQ ID NO: 26 - DNA, 348 bp, synthetic construct
  source 1..348: mol_type = other DNA
caggttcaacttcaacagtcaggcgctgaggttatgaagcctggtgcttctgtgaaagtgagctgcaaggcttctggttacaccctgaactcttactggcttgagtgggttagacagaggcctggtcatggtcttgaatggattggtgagattctgcctggtactggcagcacctcttacaaccagaagttcaagggccgagttaccttcaccgctgatacttctactaacaccgcctacatggaactgtccagccttacttctgaggacaccgctgtttactactgcgctagagaaggctggtacttcgatgtttggggtgctggtactaccgtgaccgtttcttct

SEQ ID NO: 27 - DNA, 348 bp, synthetic construct
  source 1..348: mol_type = other DNA
caggttcaacttcaacagtcaggcgctgaggttatgaagcctggtgcttctgtgaaagtgagctgcaaggcttctggttacaccctcattaggtactggcttgagtgggttagacagaggcctggtcatggtcttgaatggattggtgagatcctgcctggtagcggtggcactaattacaaccagaagttcaagggccgagtcaccttcactgctgatacttctactaacaccgcctacatggaactgtccagccttacttctgaggacaccgctgtttactactgcgctagagaaggctggtacttcgatgtttggggtgctggtactaccgtgaccgtttcttct

SEQ ID NO: 28 - protein, 17 aa, synthetic construct
EILPGSGSTNYAQKFQG

SEQ ID NO: 29 - protein, 116 aa, synthetic construct
QVQLVQSGAEVKKPGSSVKVSCKASGYTLSSYWLEWVRQAPGQGLEWIGEILPGSGSTNYAQKFQGRVTITADESTSTAYMELSSLRSEDTAVYYCAREGWYFDVWGQGTTVTVSS

SEQ ID NO: 30 - DNA, 348 bp, synthetic construct
  source 1..348: mol_type = other DNA
caggttcagcttgttcagtcaggtgctgaggttaagaagccgggctcatctgtcaaagtgagctgtaaggctagcggctacaccctgtcatcttattggcttgagtgggtgagacaagctcctggtcaaggtcttgaatggatcggtgagattctgcctggtagcggttctactaactacgcccaaaagttccagggcagagtgaccattactgctgacgagtctactagcaccgcctacatggaacttagcagccttaggtctgaggacaccgctgtttattactgcgctagagaaggctggtacttcgatgtttggggtcagggtactaccgtgaccgtttcttct

SEQ ID NO: 31 - protein, 116 aa, synthetic construct
QVQLVQSGAEVKKPGSSVKVSCKASGGTFSSYWLEWVRQRPGQGLEWIGEILPGSGSTNYNQKFQGRVTITADTSTNTAYMELSSLTSEDTAVYYCAREGWYFDVWGAGTTVTVSS

SEQ ID NO: 32 - protein, 116 aa, synthetic construct
QVQLVQSGAEVKKPGSSVKVSCKASGGTFSSYWLEWVRQAPGQGLEWIGEILPGSGSTNYAQKFQGRVTITADESTSTAYMELSSLRSEDTAVYYCAREGWYFDVWGQGTTVTVSS

SEQ ID NO: 33 - protein, 84 aa, synthetic construct
QVQLVQSGAEVKKPGSSVKVSCKASGYTLSSYWIEWVRQAPGQGLEWMGEILPGSGTANYAQKFQGRVTITADESTSTAYMELS

SEQ ID NO: 34 - protein, 112 aa, synthetic construct
DVVMTQSPLSLSVTLGQPASISCRSSQSLLYSNGNTHLNWLLQRPGQSPRRLIYLVSKLDSGVPDRFSGSGSGTDFTLKISRVEAEDVGFYYCVQGTHFPYTFGGGTKLEIK

SEQ ID NO: 35 - protein, 112 aa, synthetic construct
DVVMTQSPLSLPVTLGQPASISCRSSQSLLYSNGNTHLNWLLQRPGQSPRRLIYLVSKLDSGVPDRFSGSGSGTDFTLKISRVEAEDVGVYYCVQGTHFPYTFGGGTKLEIK

SEQ ID NO: 36 - protein, 112 aa, synthetic construct
DVVMTQSPLSLPVTLGQPASISCRSSQSLLYSNGNTHLNWLQQRPGQSPRRLIYLVSKLDSGVPDRFSGSGSGTDFTLKISRVEAEDVGVYYCVQGTHFPYTFGQGTKLEIK

SEQ ID NO: 37 - protein, 112 aa, synthetic construct
DVVMTQSPLSLPVTLGQPASISCRSSQSLLYSNGNTHLNWLLQRPGQSPRRLIYLVSKLDSGVPDRFSGSGSGTDFTLKISRVEAEDVGVYYCVQGTHFPYTFGQGTKLEIK

(19) United States
(12) Patent Application Publication
(10) Pub. No.: US 2026/0022196 A1
(43) Pub. Date: Jan. 22, 2026

(54) ANTI-FUNGAL ANTIBODIES BINDING TO BETA GLUCAN

(71) Applicant: KBIO Holdings Limited, London (GB)

(72) Inventors: Sean Stevens, London (GB); Joshua Royal, London (GB); Krystal Hamorsky, London (GB); Michael Pauly, San Diego, CA (US); Miles Brennan, San Diego, CA (US); André Frenzel, Braunschweig (DE); Jonas Kügler, Braunschweig (DE)

(73) Assignee: KBIO Holdings Limited, London (GB)

(21) Appl. No.: 18/993,466

(22) PCT Filed: Jul. 13, 2023
(86) PCT No.: PCT/GB2023/051835

§ 371 Date: Jan. 10, 2025

Related U.S. Application Data
(60) Provisional application No. 63/388,781, filed on Jul. 13, 2022.

(30) Foreign Application Priority Data
May 16, 2023 (GB) 2307285.3

(51) Int. Cl. C07K 16/44; A61K 39/00; A61K 47/68; A61P 31/04

(52) U.S. Cl. CPC C07K 16/44; A61K 47/6831; A61K 47/6879; A61K 47/6889; A61P 31/04; A61K 2039/505; C07K 2317/24; C07K 2317/31; C07K 2317/565; C07K 2317/92; C07K 2317/94

(57) ABSTRACT

The present invention relates to antibodies, or antigen-binding fragments thereof, that have binding specificity to β-glucans. Also described are the production of such monoclonal antibodies, and nucleic acids encoding such antibodies.

### FIELD OF THE INVENTION

The present invention relates to antibodies, or antigen-binding fragments thereof, that have binding specificity to β-glucans and the production of such monoclonal antibodies.

### BACKGROUND TO THE INVENTION

Antibodies are glycoproteins belonging to the immunoglobulin superfamily of proteins. There are five primary classes of immunoglobulin, including IgG, IgM, IgA, IgE and IgD. Antibodies are typically made of basic structural units comprising two heavy and light chains. The IgG immunoglobulin molecule, for example, comprises four polypeptide chains, two of which are identical heavy (H) chains and two of which are identical light (K) chains. Each heavy chain comprises an N-terminal variable domain (VH) and three constant domains (CH1, CH2 and CH3), with a hinge region linking CH1 and CH2. Each light chain comprises an N-terminal variable domain (VL) and a constant domain (CL), which together associate with the VH and CH1 domain of the heavy chain.

An antibody may recognise an antigen via the fragment antigen-binding (Fab) variable region, which comprises the VL, CL, VH and CH1 domains. The antibody may activate the immune system through the fragment crystallisable region (Fc region), which is the tail region of the antibody comprising the CH2 and CH3 domains.

The heavy chain variable region (VH) refers to the fragment of the heavy chain of an antibody that comprises the three complementarity determining regions (CDRs) which are interposed between the framework regions, the latter of which form a scaffold which supports the CDRs. Similarly, the light chain variable region (VL) comprises three CDRs and their surrounding framework regions.

The CDR, with regard to an antibody or an antigen-binding fragment thereof, is a highly variable sequence of amino acids that form a loop in the variable regions of the heavy and light chains. The CDR is therefore able to interact with the antigen. The heavy and light chain variable regions each contain three CDRs, that a referred to herein as CDRH1, CDRH2 and CDRH3 (heavy chain CDRs) and CDRL1, CDRL2 and CDRL3 (light chain CDRs).

IgG is the main antibody isotype found in blood and extracellular fluid. IgG plays many roles in the immune system, including promoting agglutination of pathogens, activating the complement system, neutralizing toxins and promoting antibody-dependent cell mediated cytotoxicity (ADCC). IgG exists as various subtypes, including IgG1, IgG2, IgG3 and IgG4. IgG1 is an exemplary antibody for the purposes of the present embodiments. IgG1 antibodies may be derived from non-human sources, such as any source outside of the human germline repertoire of antibodies. Though not identical, antibodies obtained from non-human mammals, such as mice, comprise heavy chain variable region amino acid sequences which may be typically expressed in humans.

Accordingly, sequences which are of a non-human origin but which may be expressed as though from the human antibody repertoire may be considered human sequences. When a larger portion of a non-human antibody such as IgG1 is engineered to match what is expressed in the human antibody repertoire, the engineered antibody is referred to as “humanized” and the process is referred to as “humanization”. In other respects, humanization refers to the retention of certain parts of the murine sequence, which may include the CDRs, and integration of these sequences into portions of the human antibody, such as the constant regions of the heavy and light chains. This is often also referred to as CDR-grafting, in which xenogeneic CDRs are integrated into a human antibody framework in order to reduce immunogenicity. In general, such humanized antibodies exhibit increased efficacy and a reduction in overall immunogenicity, inflammation and other undesirable effects that frequently occur in humans that are associated with murine or other non-human antibodies.

β-glucans are a heterogeneous group of natural polysaccharides found in the cell walls of various organisms, some organisms of which are pathogenic, including fungi and bacteria. Research indicates that β-glucans may play an immune-regulatory role and therefore antibodies with favourable binding affinity for β-glucans may provide beneficial results through the inhibition of these molecules.

Previously, monoclonal antibodies have been engineered with varying levels of success to provide binding to β-glucans. Even so, there remains a significant need for monoclonal antibodies that exhibit improved binding to β-glucans in clinical, diagnostic and therapeutic contexts, and particularly to humanised antibodies that exhibit these features. The embodiments described herein are directed to these and other improvements.

### SUMMARY OF THE INVENTION

The present inventors have developed an antibody, or antigen-binding fragment thereof, that targets β-glucans and may be used as an anti-fungal agent or used in the manufacture of an anti-fungal agent that may be capable of treating fungal infection.

The inventors have surprisingly demonstrated that such an antibody may be manufactured that has improved binding to β-glucans. It has been further demonstrated that, surprisingly, said antibodies result in improved clinical, therapeutic and diagnostic outcomes when used as an anti-fungal agent and/or in the treatment of fungal infection.

In one aspect, the present invention provides an antibody, or antigen binding fragment thereof, wherein the antibody or antigen-binding fragment comprises a light chain variable region comprising:
(a) a CDRL1 of SEQ ID NO 1;
(b) a CDRL2 selected from the group consisting of SEQ ID NO 2 and SEQ ID NO 3; and
(c) a CDRL3 of SEQ ID NO 4;
and a heavy chain variable region comprising:
(d) a CDRH1 selected from the group consisting of SEQ ID NOs 5-7;
(e) a CDRH2 selected from the group consisting of SEQ ID NOs 8-12 and SEQ ID NO 28; and
(f) a CDRH3 of SEQ ID NO 13.

In one aspect, the present invention provides an antibody, or antigen-binding fragment thereof, wherein the antibody, or antigen-binding fragment thereof, comprises a light chain variable region comprising an amino acid sequence having at least 70% identity to SEQ ID NO 14 and/or a heavy chain variable region comprising an amino acid sequence having at least 70% sequence identity to SEQ ID NO 16.

In one aspect, the present invention provides an antibody, or antigen-binding fragment thereof, wherein the antibody, or antigen binding fragment thereof, comprises a light chain variable region comprising an amino acid sequence having at least 70% sequence identity to SEQ ID NO 14 and/or a heavy chain variable region comprising an amino acid sequence having at least 70% sequence identity to SEQ ID NO 18.

In one aspect, the present invention provides an antibody, or antigen-binding fragment thereof, wherein the antibody, or antigen binding fragment thereof, comprises a light chain variable region comprising an amino acid sequence having at least 70% sequence identity to SEQ ID NO 15 and/or a heavy chain variable region comprising an amino acid sequence having at least 70% sequence identity to SEQ ID NO 17.

In one aspect, the present invention provides an antibody, or antigen-binding fragment thereof, wherein the antibody, or antigen binding fragment thereof, comprises a light chain variable region comprising an amino acid sequence having at least 70% sequence identity to SEQ ID NO 14 and/or a heavy chain variable region comprising an amino acid sequence having at least 70% sequence identity to SEQ ID NO 17.

In one aspect, the present invention provides an antibody, or antigen-binding fragment thereof, wherein the antibody, or antigen-binding fragment thereof, comprises a light chain variable region comprising an amino acid sequence having at least 70% sequence identity to SEQ ID NO 14 and/or a heavy chain variable region comprising an amino acid sequence having at least 70% sequence identity to SEQ ID NO 19.

In one aspect, the present invention provides an antibody, or antigen-binding fragment thereof, wherein the antibody, or antigen-binding fragment thereof, comprises a light chain variable region comprising an amino acid sequence having at least 70% sequence identity to SEQ ID NO 14 and/or a heavy chain variable region comprising an amino acid sequence having at least 70% sequence identity to SEQ ID NO 20.

In one aspect, the present invention provides an antibody, or antigen-binding fragment thereof, wherein the antibody, or antigen-binding fragment thereof, comprises a light chain variable region comprising an amino acid sequence having at least 70% sequence identity to SEQ ID NO 14 and/or a heavy chain variable region comprising an amino acid sequence having at least 70% sequence identity to SEQ ID NO 29.

In one aspect, the present invention provides an antibody, or antigen-binding fragment, wherein the antibody or antigen-binding fragment is humanized.

In one aspect, the present invention provides an antibody, or antigen-binding fragment, wherein the antibody or antigen-binding fragment is capable of binding to β-glucans.

In a further aspect, the antibody or antigen-binding fragment thereof is capable of binding to 1,3-β-glucan or a 1,6-β-glucan.

In a further aspect, the antibody, or antigen-binding fragment thereof, is capable of binding to laminarin.

In one aspect, the antibody, or antigen-binding fragment thereof, may be used as an anti-fungal agent.

In a further aspect, the antibody, or antigen-binding fragment thereof, may be used in the treatment of a fungal infection.

In a further aspect, the antibody, or antigen-binding fragment thereof, may be used in the manufacture of a medicament for the treatment of fungal infection.

In a further aspect, the antibody, or antigen-binding fragment thereof, may be used in the treatment of an Aspergillus infection.

In a further aspect, the antibody, or antigen-binding fragment thereof, may be used in the treatment of an Aspergillus fumigatus infection.

In a further aspect, the present invention provides a method of treating a fungal infection comprising administering the antibody, or antigen-binding fragment thereof, of the present invention.

In a further aspect, the present invention provides a method of treating an Aspergillus infection, comprising administering the antibody, or antigen-binding fragment thereof, of the present invention.

In a further aspect, the present invention provides a method of treating an Aspergillus fumigatus infection, comprising administering the antibody, or antigen-binding fragment thereof, of the present invention.

In one aspect, the present invention provides a nucleic acid that encodes an antibody, or antigen-binding fragment thereof, as disclosed herein.

In a further aspect, the present invention provides a nucleic acid encoding a light chain variable region having at least 70% sequence identity to SEQ ID NO 21 and/or a heavy chain variable region having at least 70% sequence identity to SEQ ID NO 23.

In a further aspect, the present invention provides a nucleic acid encoding a light chain variable region having at least 70% sequence identity to SEQ ID NO 21 and/or a heavy chain variable region having at least 70% sequence identity to SEQ ID NO 24.

In a further aspect, the present invention provides a nucleic acid encoding a light chain variable region having at least 70% sequence identity to SEQ ID NO 22 and/or a heavy chain variable region having at least 70% sequence identity to SEQ ID NO 24.

In a further aspect, the present invention provides a nucleic acid encoding a light chain variable region having at least 70% sequence identity to SEQ ID NO 21 and/or a heavy chain variable region having at least 70% sequence identity to SEQ ID NO 25.

In a further aspect, the present invention provides a nucleic acid encoding a light chain variable region having at least 70% sequence identity to SEQ ID NO 21 and/or a heavy chain variable region having at least 70% sequence identity to SEQ ID NO 26.

In a further aspect, the present invention provides a nucleic acid encoding a light chain variable region having at least 70% sequence identity to SEQ ID NO 21 and/or a heavy chain variable region having at least 70% sequence identity to SEQ ID NO 27.

In a further aspect, the present invention provides a nucleic acid encoding a light chain variable region having at least 70% sequence identity to SEQ ID NO 21 and/or a heavy chain variable region having at least 70% sequence identity to SEQ ID NO 30.

In one aspect, the present invention provides a nucleic acid encoding an antibody, or antigen-binding fragment, wherein the antibody or antigen-binding fragment is humanized.

In one aspect, the present invention provides a nucleic acid encoding an antibody, or antigen-binding fragment, wherein the antibody or antigen-binding fragment is capable of binding to β-glucans.

In a further aspect, the nucleic acid encodes an antibody, or antigen-binding fragment thereof, that is capable of binding to 1,3-β-glucan or a 1,6-β-glucan.

In a further aspect, the nucleic acid encodes an antibody, or antigen-binding fragment thereof, that is capable of binding to laminarin.

### DESCRIPTION OF DRAWINGS

The drawings and embodiments described herein are illustrative of multiple alternative structures, aspects, and features of the multiple embodiments and alternatives disclosed herein, and they are not to be understood as limiting the scope of any of these embodiments and alternatives.

FIG. 1: Laminarin binding (ELISA) of the initial humanized and next generation affinity matured anti-fungal antibodies

FIG. 1 is a graph of ELISA data for EC50 binding of the initial humanized anti-fungal antibodies, clones 3.1, 14.1 and 32.3 (KB14A.3.1, KB14A. 14.1 and KB14A.32.3), and the next generation, humanized and affinity-matured anti-fungal antibodies using laminarin, namely clones 39.1, 40.1, 41.2, 42.1 and 43.1 (KB14A.39.1, KB14A.40.1, KB14A.41.2, KB14A.42.1 and KB14A.43.1) using laminarin.

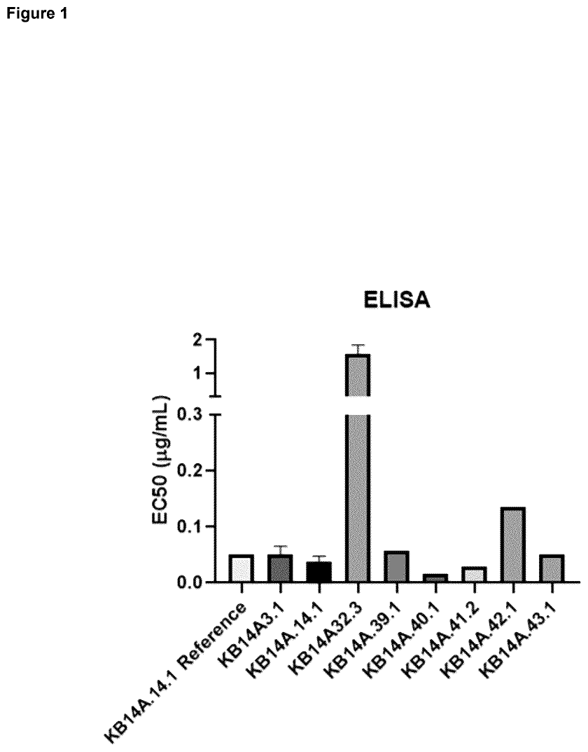

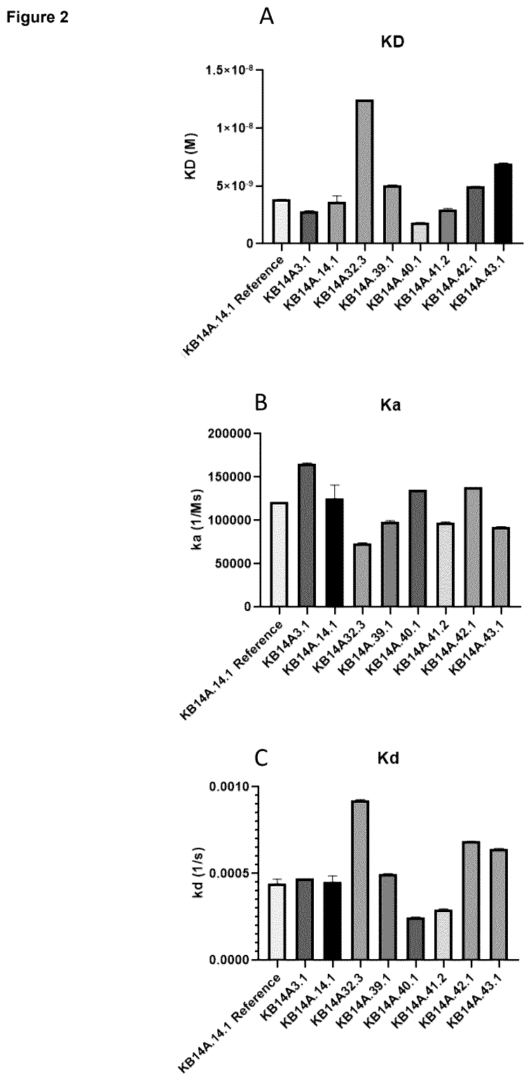

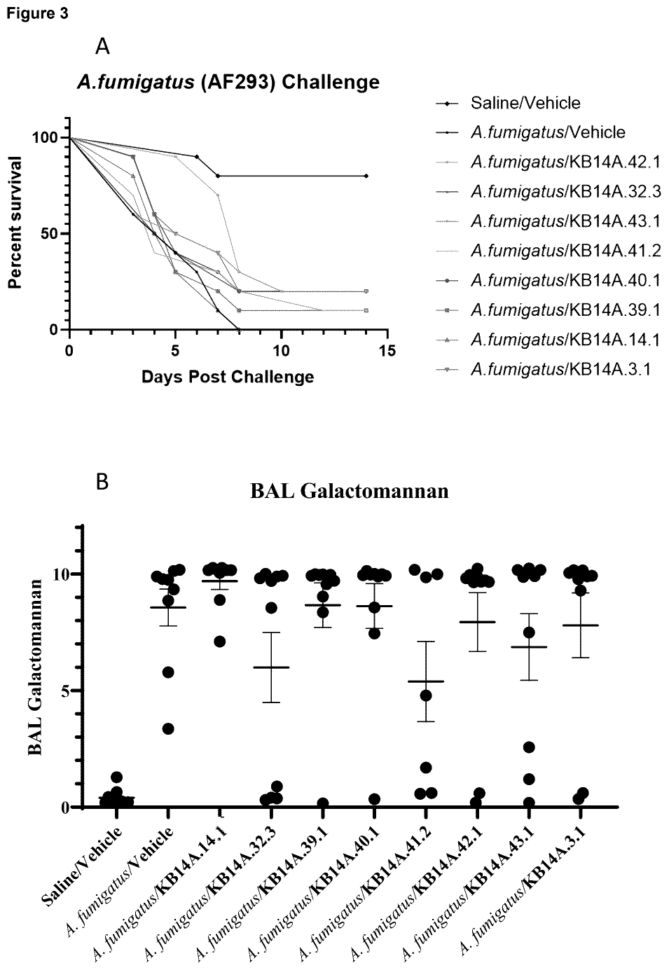

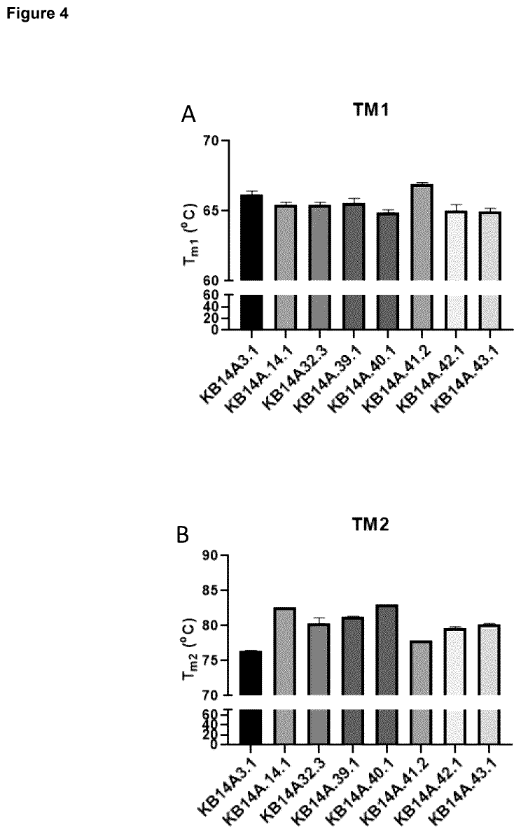

It can be seen from the graph that clones 3.1 and 14.1 have an EC50 value of less than 0.1 μg/mL and that clone 32.3 has an EC50 value of less than 2.5 μg/mL (FIG. 1. All values plotted represent the mean value+/−the standard deviation (SD). Specifically, clone 3.1 has an EC50 of 0.05124 μg/mL, KB14A. 14.1 has an EC50 of 0.037125 μg/mL, and clone 32.3 has an EC50 of 1.5845 μg/mL (Table 5).

It can be seen from the graph that clones 39.1, 40.1, 41.2 and 43.1 have an EC50 value of less than 0.1 μg/mL (FIG. 1). Clone 42.1 has an EC50 value of less than 0.2 μg/mL (FIG. 1). All values plotted represent the mean value. Specifically, clone 39.1 has an EC50 of 0.05649 μg/mL, clone 40.1 has an EC50 of 0.017 μg/mL, clone 41.2 has an EC50 of 0.02878, clone 42.1 has an EC50 of 0.1356 μg/mL, and clone 43.1 has an EC50 of 0.05045 μg/mL (Table 5).

FIG. 2: Laminarin binding affinity (K_{D}) of initial humanized and next generation affinity matured anti-fungal antibodies

FIG. 2 is a series of graphs displaying the Kp values (FIG. 2A), ka values (FIG. 2B) and kd values (FIG. 2C) for each of the initial humanized anti-fungal antibodies, clones 3.1, 14.1, and 32.3, and for each of the next-generation, humanized and affinity matured antibody clones 39.1, 40.1, 41.2, 42.1, and 43.1 using laminarin Biocore analysis. All values plotted represent the mean value+/−the standard deviation (SD).

Specific values for the K_{D}, ka and kd of each antibody detailed in the corresponding tables (Tables 6-8).

It can be seen from the graph and corresponding table that clone 3.1 and clone KB14A. 14.1 have a Kp of less than 5×10⁻⁹ and clone 32.3 has a Kp value of less than 1.5×10⁻⁸ (FIG. 2A; Table 8).

It can be seen from the graph and corresponding table that clones 41.2, 40.1 and 42.1 all have a K_{D} value of 5×10⁻⁹ or less. Clones 39.1 and 43.1 have a K_{D} of less than 1×10⁻⁸ (FIG. 2A; Table 8).

FIG. 3: Improved survival of immunosuppressed mice infected with Aspergillus fumigatus following treatment with the initial humanized anti-fungal antibody or the next-generation, humanized and affinity matured antibody

FIG. 3A is a graph showing the % survival of immunosuppressed mice following challenge with Aspergillus fumigatus (AF293). Test groups were given a 15 mg/kg dose of an initial humanised antibody (clones 3.1, 14.1 or 32.3) or a next-generation, humanized and affinity matured antibody (clones 39.1, 40.1, 41.2, 42.1, and 43.1).

Mice treated with two IV administrations of the initial humanized antibody or the next-generation, humanized and affinity matured antibody show improved survival over the vehicle control treated mice (FIG. 3A).

FIG. 3B details the level of fungi in the lungs of each test group following saline control, A. fumigatus/Vehicle control or antibody treatment, through measure of Bronchoalveolar Lavage (BAL) Galactomannan levels. Galactomannan, which is, for example, a major component of the Aspergillus cell wall, may act as an indicator of fungal burden in a host due to its release during infection.

The data show that for the A. fumigatus/Vehicle group and for each antibody treated group that a significant number of animals display detectable levels of fungi in the lungs through measure of BAL Galactomannan levels. The saline/vehicle control, as expected, did not show detectable levels of fungi in the lungs. Furthermore, Galactomannan levels in Aspergillus challenged mice correlated with survival.

FIG. 4: Thermal shift assay (TSA) of the initial humanized and next generation affinity matured anti-fungal antibodies

FIG. 4 details the thermostability of each antibody clone. The melting temperatures (Tm) of each antibody were determined by the TSA performed on a Life Technologies qPCR system. The Tm1 of each antibody is detailed in FIG. 4A, with precise values provided in Table 9. The Tm1 value represents the first transition which is thought to involve the thermal unfolding of the CH2 domain. The Tm2 of each antibody is detailed in FIG. 4B, with precise values provided in Table 10. The Tm2 value represents a second transition which is representative of the unfolding of the CH3/Fab domain.

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation and amino acid sequences are written left to right in amino to carboxy orientation, respectively.

The publications discussed herein are provided solely for their disclosure prior to the filing date of the present application. Nothing herein is to be construed as an admission that such publications constitute prior art to the claims appended hereto. All publications mentioned in the specification are herein incorporated by reference.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

### Sequence of Antibody or Antigen-Binding Fragment Thereof

In one aspect, the invention provides an antibody, or an antigen-binding fragment thereof, wherein the antibody or antigen-binding fragment thereof comprise a light chain variable region comprising:
(a) a CDRL1 of SEQ ID NO 1;
(b) a CDRL2 selected from the group consisting of SEQ ID NO 2 and SEQ ID NO 3; and
(c) a CDRL3 of SEQ ID NO 4;
and a heavy chain variable region comprising:
(d) a CDRH1 selected from the group consisting of SEQ ID NOs 5-7;
(e) a CDRH2 selected from the group consisting of SEQ ID NOs 8-12 and SEQ ID NO 28; and
(f) a CDRH3 of SEQ ID NO 13.

In one embodiment, the antibody, or antigen binding-fragment thereof, of the present in invention may comprise a light chain variable region comprising three light chain variable region CDRs selected from a CDRL1 of SEQ ID NO 1, a CDRL2 of SEQ ID NO 2 or SEQ ID NO 3, and a CDRL3 of SEQ ID NO 4 and three heavy chain variable region CDRs selected from a CDRH1 of SEQ ID NO 5, SEQ ID NO 6, or SEQ ID NO 7, a CDRH2 selected from SEQ ID NO 8, SEQ ID NO 9, SEQ ID NO 10, SEQ ID NO 11, SEQ ID NO 12, or SEQ ID NO 28 and a CDRH3 of SEQ ID NO 13.

The present invention includes “variants” of the CDR regions described below. A “variant” of any given sequence is a sequence in which the specific sequence of residues (whether amino acid or nucleic acid residues) has been modified in such a manner that the polypeptide or polynucleotide in question retains at least one or all of its endogenous functions. A variant sequence can be obtained by addition, deletion, substitution, modification, replacement and/or variation of at least one residue present in the naturally occurring polypeptide or polynucleotide.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 2 or a variant thereof; and
(c) a CDRL3 of SEQ ID NO 4 or a variant thereof;
and a heavy chain variable region comprising:
(d) a CDRH1 of SEQ ID NO 5 or a variant thereof;
(e) a CDRH2 of SEQ ID NO 8 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 16.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 16.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 wherein the amino acid sequence comprises a CDRL1 of SEQ ID NO 1 or a variant thereof, a CDRL2 of SEQ ID NO 2 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 16 wherein the amino acid sequence comprises a CDRH1 of SEQ ID NO 5 or a variant thereof, a CDRH2 of SEQ ID NO 8 or a variant thereof and a CDRH3 of SEQ ID NO 13 or a variant thereof.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 2 or a variant thereof; and
(c) a CDRL3 of SEQ ID NO 4 or a variant thereof;
and a heavy chain variable region comprising:
(d) a CDRH1 of SEQ ID NO 5 or a variant thereof;
(e) a CDRH2 of SEQ ID NO 10 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 18.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 18.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 wherein the amino acid sequence comprises a CDRL1 of SEQ ID NO 1 or a variant thereof, a CDRL2 of SEQ ID NO 2 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 18 wherein the amino acid sequence comprises a CDRH1 of SEQ ID NO 5 or a variant thereof, a CDRH2 of SEQ ID NO 10 or a variant thereof and a CDRH3 of SEQ ID NO 13 or a variant thereof.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 3 or a variant thereof; and
(c) a CDRL3 of SEQ ID NO 4 or a variant thereof;
and a heavy chain variable region comprising:
(d) a CDRH1 of SEQ ID NO 6 or a variant thereof;
(e) a CDRH2 of SEQ ID NO 9 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 15.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 17.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 15 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 17.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 15 wherein the amino acid sequence comprises a CDRL1 of SEQ ID NO 1 or a variant thereof, a CDRL2 of SEQ ID NO 3 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 17 wherein the amino acid sequence comprises a CDRH1 of SEQ ID NO 6 or a variant thereof, a CDRH2 of SEQ ID NO 9 or a variant thereof and a CDRH3 of SEQ ID NO 13 or a variant thereof.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 2 or a variant thereof; and
(c) a CDRL3 of SEQ ID NO 4 or a variant thereof;
and a heavy chain variable region comprising:
(d) a CDRH1 of SEQ ID NO 6 or a variant thereof;
(e) a CDRH2 of SEQ ID NO 9 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 17.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 17.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 wherein the amino acid sequence comprises a CDRL1 of SEQ ID NO 1 or a variant thereof, a CDRL2 of SEQ ID NO 2 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 17 wherein the amino acid sequence comprises a CDRH1 of SEQ ID NO 6 or a variant thereof, a CDRH2 of SEQ ID NO 9 or a variant thereof and a CDRH3 of SEQ ID NO 13 or a variant thereof.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 2 or a variant thereof; and
(c) a CDRL3 of SEQ ID NO 4 or a variant thereof;
and a heavy chain variable region comprising:
(d) a CDRH1 of SEQ ID NO 5 or a variant thereof;
(e) a CDRH2 of SEQ ID NO 11 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 19.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 19.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 wherein the amino acid sequence comprises a CDRL1 of SEQ ID NO 1 or a variant thereof, a CDRL2 of SEQ ID NO 2 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 19 wherein the amino acid sequence comprises a CDRH1 of SEQ ID NO 5 or a variant thereof, a CDRH2 of SEQ ID NO 11 or a variant thereof and a CDRH3 of SEQ ID NO 13 or a variant thereof.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 2 or a variant thereof; and
(c) a CDRL3 of SEQ ID NO 4 or a variant thereof;
and a heavy chain variable region comprising:
(d) a CDRH1 of SEQ ID NO 7 or a variant thereof;
(e) a CDRH2 of SEQ ID NO 12 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 20.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 20.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 wherein the amino acid sequence comprises a CDRL1 of SEQ ID NO 1 or a variant thereof, a CDRL2 of SEQ ID NO 2 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 20 wherein the amino acid sequence comprises a CDRH1 of SEQ ID NO 7 or a variant thereof, a CDRH2 of SEQ ID NO 12 or a variant thereof and a CDRH3 of SEQ ID NO 13 or a variant thereof.

In one embodiment, the antibody or antigen-binding fragment thereof comprises a light chain variable region comprising:
(a) a CDRL1 of SEQ ID NO 1 or a variant thereof;
(b) a CDRL2 of SEQ ID NO 2 or a variant thereof; and
(c) a CDRL3 of SEQ ID NO 4 or a variant thereof;
and a heavy chain variable region comprising:
(d) a CDRH1 of SEQ ID NO 6 or a variant thereof;
(e) a CDRH2 of SEQ ID NO 28 or a variant thereof; and
(f) a CDRH3 of SEQ ID NO 13 or a variant thereof.

In one embodiment, the antibody or antigen-binding fragment thereof comprises a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14.

In one embodiment, the antibody or antigen-binding fragment thereof comprises a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 29.

In one embodiment, the antibody or antigen-binding fragment thereof comprises a light chain variable region and a heavy chain variable region, wherein the light chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 and the heavy chain variable region comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 29.

In one embodiment, the antibody or antigen-binding fragment thereof comprises or consists of:
(i) a light chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 14 wherein the amino acid sequence comprises a CDRL1 of SEQ ID NO 1 or a variant thereof, a CDRL2 of SEQ ID NO 2 or a variant thereof and a CDRL3 of SEQ ID NO 4 or a variant thereof; and/or
(ii) a heavy chain variable region comprising an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO 29 wherein the amino acid sequence comprises a CDRH1 of SEQ ID NO 6 or a variant thereof, a CDRH2 of SEQ ID NO 28 or a variant thereof and a CDRH3 of SEQ ID NO 13 or a variant thereof.

### Additional Features of Antibody or Antigen-Binding Fragment Thereof

In one embodiment, the antibody, or antigen-binding fragment thereof, may be IgG and all isotypes thereof.

In one embodiment the antibody, or antigen-binding fragment thereof, may be IgG1 and all allotypes thereof.

In one embodiment the antibody, or antigen-binding fragment thereof, may be IgG2 and all allotypes thereof.

In one embodiment the antibody, or antigen-binding fragment thereof, may be IgG3 and all allotypes thereof.

In one embodiment the antibody, or antigen-binding fragment thereof, may be IgG4 and all allotypes thereof.

In one embodiment the antibody, or antigen-binding fragment thereof, may be IgA and all isotypes thereof.

In one embodiment the antibody, or antigen-binding fragment thereof, may be IgA1 and allotypes thereof.

In one embodiment the antibody, or antigen-binding fragment thereof, may be IgA2 and all allotypes thereof.

In one embodiment the antibody, or antigen-binding fragment thereof, may be IgM and all allotypes thereof.

In one embodiment the antibody, or antigen-binding fragment thereof, may be IgE and all allotypes thereof.

In one embodiment the antibody, or antigen-binding fragment thereof, may be IgD and all isotypes thereof.

In one embodiment the antibody, or antigen-binding fragment thereof, may be IgD1 and all allotypes thereof.

In one embodiment the antibody, or antigen-binding fragment thereof, may be IgD2 and all allotypes thereof.

In one embodiment, the antibody, or antigen-binding fragment thereof is a chimeric antibody.

In a preferred embodiment, the antibody, or antigen-binding fragment thereof, is a humanized antibody.

In a preferred embodiment, the antibody, or antigen-binding fragment thereof is an affinity-matured antibody.

In a preferred embodiment, the antibody, or antigen-binding fragment thereof, is an affinity-matured, humanized antibody.

Accordingly, the terms “humanized”, “humanization”, or other forms related to the term “humanize” when used in the context of this disclosure shall be understood as referring to an antibody structure, which may be a complete antibody or any portion or fragment thereof, which are modified to closely resemble the human IgG1 but retaining at least one CDR of non-human origin. Such modifications may be performed on a murine IgG antibody, for example IgG1, and in some embodiments, the subject monoclonal antibodies are humanized or chimeric. Such modifications can be characterized by a substitution of one or more amino acids consistent with a human germline sequence for a corresponding number of amino acids found in a non-human mammalian form of the IgG1. Such substitutions may occur in one or more of the CDRs or within the framework regions between the CDRs, or indeed anywhere on the light and heavy variable chains.

### Antibody Fragments

Whilst the disclosure herein discusses IgG at greater length, the scope of embodiments is not limited to IgG, but includes other naturally occurring antibodies, such as IgM, IgA, IgE and IgD and their respective subtypes and allotypes. The scope of embodiments further includes antigen-binding fragments of said antibody, including but not limited to an antigen-binding fragment (Fab), a fragment antibody (F(ab′)₂), single chain antibody (scFv) and a single-domain antibody (sdAb).

A fragment antibody (F(ab′)2), refers to a region on an antibody that remains following digestion of the Fc region which leaving intact portions of the hinge region.

A single chain antibody (scFv) refers to an antibody that has been engineered to consist of a light and heavy chain variable region that are connected by a peptide-linker sequence. The peptide linker sequence is typically in the length of 10-25 amino acids, rich in glycine for flexibility and rich in serine or threonine for solubility. The peptide linker may connect the N-terminus of the heavy chain variable region with the C-terminus of the light chain variable region.

A single domain antibody (sdAb), which is often referred to as a nanobody, refers to an antigen-binding fragment of an antibody that consists of a single monomeric variable antibody domain. Therefore, an sdAb may be a light chain variable region or a heavy chain variable region. Examples of single-domain antibodies include, but are not limited to, VHH fragments and VNAR fragments. VHH and VNAR fragments comprise the antigen-binding fragment of the heavy chain.

In one embodiment, the antigen-binding fragment of the antibodies disclosed herein may be any fragment of the antibodies disclosed herein.

In another embodiment the antigen-binding fragment of the antibodies disclosed herein may be a genetically-engineered product of one or more of the fragments of the antibody.

Whilst the disclosure herein discusses IgG at greater length, the scope of embodiments is not limited to IgG, but includes other naturally occurring antibodies, such as IgM, IgA, IgE and IgD and their respective subtypes.

In one embodiment the fragment is an antigen-binding fragment (Fab), a fragment antibody (F(ab′)₂), a single-chain variable fragment (scFV) or a single domain antibody (sdAb), or a camelid antibody (VHH).

The antibodies, or antibody-binding fragments thereof, disclosed herein may be combined to produce an engineered antigen binding protein that displays specificity to two or more different antigens or epitopes. Further, the antibodies, or antigen-binding fragments thereof, disclosed herein may be combined with alternative antibodies to produce an engineered antigen binding protein that displays specificity to two or more different antigens or epitopes. Further, the antibodies, or antigen-binding fragments thereof, disclosed herein may be combined with one or more identical antibodies, antigen-binding fragments thereof, in order to form a homo-dimer or homo-multimer.

### Binding Properties of Antibodies

In one embodiment, the antibody, or antigen-binding fragment thereof, disclosed herein binds to β-glucan.

In a further embodiment, the antibody, or antigen-binding fragment thereof, disclosed herein binds to 1,3-β-glucan or a 1,6-β-glucan.

In a further embodiment, the antibody or antigen-binding fragment thereof, disclosed herein binds to laminarin.

The antibody, or antigen-binding fragment thereof, disclosed herein may be bispecific. The term bispecific is used to denote an antibody-based molecule, such as an antibody, or antigen-binding fragment thereof, or a combination of such, that has two different antigen-binding sites. In other words, it may be an engineered antibody that engages two targets or two epitopes on the same target. Bispecific antibodies come in many formats. For example, a bispecific antibody may be generated through the combination of any of the antibodies, or antigen-binding fragments thereof, disclosed herein with another antigen-binding protein, antibody, or antigen-binding fragment thereof.

The antibody, or antigen-binding fragment thereof, disclosed herein may be multi-specific. The term multi-specific is used to denote an antibody-based molecule, such as an antibody, or antigen-binding fragment thereof, or a combination of such, that has two or more different antigen-binding sites. In other words, it may be an engineered antibody that engages two or more targets, or two or more epitopes on the same target. Multi-specific antibodies come in many formats. For example, a multi-specific antibody may be generated through the combination of at least one of the antibodies, or antigen-binding fragments thereof, disclosed herein and at least one other antigen-binding protein, antibody, or antigen-binding fragment thereof.

In one embodiment, the antibody, or antigen-binding fragment thereof, disclosed herein, may be used in a bispecific antigen-binding protein, antibody, or antigen-binding fragment thereof, that binds to two different antigens or epitopes.

In a further embodiment, the antibody, or antigen-binding fragment thereof, disclosed herein, may be used in a bispecific antigen-binding protein, antibody, or antigen-binding fragment thereof, that binds to β-glucan and an additional antigen or epitope.

In a further embodiment, the antibody, or antigen-binding fragment thereof, disclosed herein, may be used in a bispecific antigen-binding protein, antibody, or antigen-binding fragment thereof, that binds to a 1,3-β-glucan or a 1,6-β-glucan and an additional antigen or epitope.

In a further embodiment, the antibody, or antigen-binding fragment thereof, disclosed herein, may be used in a bispecific antigen-binding protein, antibody, or antigen-binding fragment thereof, that binds to laminarin and an additional antigen or epitope.

In one embodiment, the antibody, or antigen-binding fragment thereof, disclosed herein, may be used in a multi-specific antigen-binding protein, antibody, or antigen-binding fragment thereof, that binds to two or more different antigens or epitopes.

In a further embodiment, the antibody, or antigen-binding fragment thereof, disclosed herein, may be used in a multi-specific antigen-binding protein, antibody, or antigen-binding fragment thereof, that binds to two or more different antigens or epitopes.

In a further embodiment, the antibody, or antigen-binding fragment thereof, disclosed herein, may be used in a multi-specific antigen-binding protein, antibody, or antigen-binding fragment thereof, that binds to β-glucan and one or more different antigens or epitopes.

In a further embodiment, the antibody, or antigen-binding fragment thereof, disclosed herein, may be used in a multi-specific antigen-binding protein, antibody, or antigen-binding fragment thereof, that binds to a 1,3-β-glucan or a 1,6-β-glucan and one or more different antigens or epitopes.

In a further embodiment, the antibody, or antigen-binding fragment thereof, disclosed herein, may be used in a multi-specific antigen-binding protein, antibody, or antigen-binding fragment thereof, that binds to laminarin and one or more different antigens or epitopes.

In another embodiment, the antibody, or antigen-binding fragment thereof, disclosed herein, may be in the form of a homo-dimer.

A homo-dimer, by definition, may be formed through the linkage or interaction of two of the same protein. As such, in one embodiment, the antibody, or antigen-binding fragment thereof, disclosed herein, may be linked or interact with another identical antibody or antigen-binding fragment thereof to produce a homo-dimer.

In one embodiment, the antibody, or antigen-binding fragment thereof, disclosed herein, may be in the form of a homo-dimer composed of two antibody molecules, or antigen-binding fragments thereof, having the same antigen binding specificity.

In one embodiment, the antibody, or antigen-binding fragment thereof, disclosed herein, may be in the form of a homo-dimer composed of two antibody molecules, or antigen binding fragments thereof, having the same antigen-binding specificity to β-glucan.

In one embodiment, the antibody, or antigen-binding fragment thereof, disclosed herein, may be in the form of a homo-dimer composed of two antibody molecules, or antigen-binding fragments thereof, having the same antigen binding specificity to a 1,3-β-glucan or a 1,6-β-glucan.

In one embodiment, the antibody, or antigen-binding fragment thereof, disclosed herein, may be in the form of a homo-dimer composed of two-antibody molecules, or antigen binding fragments thereof, having the same antigen binding specificity to laminarin.

In another embodiment, the antibody, or antigen-binding fragment thereof, disclosed herein, may be in form of a homo-multimer.

A homo-multimer, by definition, may be formed through the linkage or interaction of two or more of the same protein. As such, in one embodiment, the antibody, or antigen-binding fragment thereof, disclosed herein, may be linked or interact with one or more identical antibodies or antigen-binding fragments thereof to produce a homo-multimer.

In one embodiment, the antibody, or antigen-binding fragment thereof, disclosed herein, may be in the form of a homo-multimer composed of two or more antibody molecules, or antigen-binding fragments thereof, having the same antigen binding specificity.

In one embodiment, the antibody, or antigen-binding fragment thereof, disclosed herein, may be in the form of a homo-multimer composed of two or more antibody molecules, or antigen-binding fragments thereof, having the same antigen binding specificity to β-glucan.

In one embodiment, the antibody, or antigen-binding fragment thereof, disclosed herein, may be in the form of a homo-multimer composed of two or more antibody molecules, or antigen-binding fragments thereof, having the same antigen binding specificity to a 1,3-β-glucan or a 1,6-β-glucan.

In one embodiment, the antibody, or antigen-binding fragment thereof, disclosed herein, may be in the form of a homo-multimer composed of two or more antibody molecules, or antigen-binding fragments thereof, having the same antigen binding specificity to laminarin.

In one embodiment, the homo-dimer or homo-multimer displays increased avidity. In other words, the strength of the interaction between the homo-dimer or homo-multimer and the antigen is increased.

The antibody of the invention, or antigen-binding fragment thereof, may be defined by its EC50 value. The EC50 value is a measure of the half-maximal effective concentration, indicating the concentration of a drug required to have half the maximum effect. The binding of the antibodies, or antigen-binding fragments thereof, disclosed herein may therefore be represented through their EC50 value as determined by any suitable assay, such as an ELISA. Suitably, an antibody, or antigen-binding fragment thereof, disclosed herein, may have an EC50 value of about 2 μg/mL or less, about 1 μg/mL or less, about 0.5 μg/mL or less, about 0.4 μg/mL or less, about 0.3 μg/mL or less, about 0.2 μg/mL or less, about 0.1 μg/mL or less, or about 0.05 μg/mL or less, as determined by laminarin ELISA. Antibody binding affinity may also be determined by equilibrium binding constants (K_{D}), which may be determined by a suitable assay. Suitably, an antibody, or antigen-binding fragment thereof, may bind to a β-glucan, such as 1,3-β-glucan, 1,6-3-glucan or laminarin, with a binding affinity (K_{D}) of 1.5×10⁻⁸ M or less, 1×10⁻⁸ M or less, 9×10⁻⁹ M or less, 8×10⁻⁹ M or less, 7×10⁻⁹ M or less, 6×10⁻⁹ M or less, 5×10⁻⁹ M or less, 4×10⁻⁹ M or less, 3×10⁻⁹ M or less, 2×10⁻⁹ M or less, or 1×10⁻⁹ M or less.

The antibody, or antigen-binding fragment thereof, may be further defined by its thermostability. Thermostability may be defined by the unfolding events that occur as the antibody, or antigen-binding fragment thereof, experiences increasing temperatures. ‘Tonset’ is the temperature at which unfolding events begin to occur, ‘Tm’ is the temperature at the midpoint of the unfolding events, and Tagg is the temperature at which the unfolding events have resulted in aggregation. ‘Tm’, the midpoint of unfolding events, usually occurs as two transitions referred to as Tm1 and Tm2. The Tm1 value represents the first transition which is thought to involve the thermal unfolding of the CH2 domain. The Tm2 value represents a second transition which is representative of the unfolding of the CH3/Fab domain.

In one embodiment, the antibody, or antigen-binding fragment thereof, is thermostable up 60° C.

In one embodiment, the antibody, or antigen-binding fragment thereof, is thermostable up 70° C.

In one embodiment, the antibody, or antigen-binding fragment thereof, is thermostable up 80° C.

### Pharmaceutical Formulations

In one embodiment, the present invention provides a pharmaceutical composition comprising the antibody, or antigen-binding fragment thereof, of the present invention. The antibody, or antigen-binding fragment thereof may be in combination with a pharmaceutically acceptable carrier, diluent, excipient or adjuvant.

In one embodiment, the present invention provides a pharmaceutical composition comprising the antibody, or antigen-binding fragment thereof, in combination with L-histidine and sucrose.

In one embodiment, the present invention provides a pharmaceutical composition comprising at 5 mg/kg of the antibody, or antigen-binding fragment thereof, in combination with 20 mM L-histidine and 240 mM sucrose.

In one embodiment, the present invention provides a pharmaceutical composition comprising at least 1 mg/g, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/k, at least 15 mg/kg or at least 20 mg/kg of the antibody, or antigen-binding fragment thereof, in combination with at least 5 mM, at least 10 mM, at least 15 mM, at least 16 mM, at least 17 mM, at least 18 mM, at least 19 mM, at least 20 mM, at least 21 mM, at least 22 mM, at least 23 mM, at least 24 mM, at least 25 mM, or at least 30 mM L-histidine and at least 200 mM, at least 210 mM, at least 220 mM, at least 230 mM, at least 235, at least 236 mM, at least 237 mM, at least 238 mM, at least 239 mM, at least 240 mM, at least 241 mM, at least 242 mM, at least 243 mM, at least 244 mM, at least 245 mM, at least 250 mM, at least 260 mM, at least 270 mM, at least 280 mM, at least 290 mM, or at least 230 mM sucrose.

In one embodiment, the present invention provides an antibody, or antigen-binding fragment thereof, according to the present invention and/or a pharmaceutical composition according to the present invention, for use as a medicament.

In one embodiment, the present invention provides use of an antibody, or antigen-binding fragment thereof, according to the present invention, or a pharmaceutical composition according to the present invention, for the manufacture of a medicament.

In one embodiment, the present invention provides a method comprising administering an antibody, or antigen-binding fragment thereof, according to the present invention, or a pharmaceutical composition according to the present invention, to a subject in need thereof.

In one embodiment, the antibody, or antigen-binding fragment thereof, of the present invention may be used as an anti-fungal agent.

In one embodiment, the pharmaceutical composition comprising the antibody, or antigen-binding fragment thereof, may be used as an anti-fungal agent.

In one embodiment, the antibody, or antigen-binding fragment thereof, of the present invention may be used in the treatment of fungal infection.

In one embodiment, the pharmaceutical composition comprising the antibody, or antigen-binding fragment thereof, may be used in the treatment of fungal infection.

In one embodiment, the antibody, or antigen-binding fragment thereof, or pharmaceutical composition comprising the antibody, or antigen-binding fragment thereof, may be used in the treatment of an Aspergillus infection.

In one embodiment, the antibody, or antigen-binding fragment thereof, or pharmaceutical composition comprising the antibody, or antigen-binding fragment thereof, may be used in the treatment of an Aspergillus fumigatus infection.

In one embodiment, the antibody, or antigen-binding fragment thereof, or pharmaceutical composition comprising the antibody, or antigen-binding fragment thereof, may be used in the treatment of a Candida infection.

In one embodiment, the antibody, or antigen-binding fragment thereof, or pharmaceutical composition comprising the antibody, or antigen-binding fragment thereof, may be used in the treatment of a Candida albicans or Candida auris infection.

In one embodiment, the present invention provides a method of treatment of a fungal infection comprising administering the antibody, or antigen-binding fragment thereof.

In one embodiment, the present invention provides a method of treatment of a fungal infection comprising administering the pharmaceutical composition comprising the antibody, or antigen-binding fragment thereof.

In one embodiment, the present invention provides a method of treatment of an Aspergillus infection comprising administering the antibody, or antigen-binding fragment thereof, or pharmaceutical composition comprising the antibody, or antigen-binding fragment thereof.

In one embodiment, the present invention provides a method of treatment of an Aspergillus fumigatus infection comprising administering the antibody, or antigen-binding fragment thereof, or pharmaceutical composition comprising the antibody, or antigen-binding fragment thereof.

In one embodiment, the antibody, or antigen-binding fragment thereof, of the present invention may be used in the manufacture of a medicament for the treatment of fungal infection.

In one embodiment, the pharmaceutical composition comprising the antibody, or antigen-binding fragment thereof, of the present invention may be used in the manufacture of a medicament for the treatment of fungal infection.

In one embodiment, the antibody, or antigen-binding fragment thereof, or pharmaceutical composition comprising the antibody, or antigen-binding fragment thereof, of the present invention may be used in the manufacture of a medicament for the treatment of an Aspergillus infection.

In one embodiment, the antibody, or antigen-binding fragment thereof, or pharmaceutical composition comprising the antibody, or antigen-binding fragment thereof, of the present invention may be used in the manufacture of a medicament for the treatment of an Aspergillus fumigatus infection.

In one embodiment, the present invention provides a method of treatment of an Candida infection comprising administering the antibody, or antigen-binding fragment thereof, or pharmaceutical composition comprising the antibody, or antigen-binding fragment thereof.

In one embodiment, the present invention provides a method of treatment of an Candida albicans infection comprising administering the antibody, or antigen-binding fragment thereof, or pharmaceutical composition comprising the antibody, or antigen-binding fragment thereof.

In one embodiment, the present invention provides a method of treatment of an Candida auris infection comprising administering the antibody, or antigen-binding fragment thereof, or pharmaceutical composition comprising the antibody, or antigen-binding fragment thereof.

In one embodiment, the antibody, or antigen-binding fragment thereof, or pharmaceutical composition comprising the antibody, or antigen-binding fragment thereof, of the present invention may be used in the manufacture of a medicament for the treatment of an Candida infection.

In one embodiment, the antibody, or antigen-binding fragment thereof, or pharmaceutical composition comprising the antibody, or antigen-binding fragment thereof, of the present invention may be used in the manufacture of a medicament for the treatment of an Candida albicans infection.

In one embodiment, the antibody, or antigen-binding fragment thereof, or pharmaceutical composition comprising the antibody, or antigen-binding fragment thereof, of the present invention may be used in the manufacture of a medicament for the treatment of an Candida auris infection.

### Antibody-Drug Conjugates

Antibody-drug conjugates (ADCs) are therapeutic molecules that may be specifically targeted to an antigen via the antibody, or an antigen-binding fragment thereof, enabling precise delivery of a drug to a specific location. For example, ADCs have been used as chemotherapeutic agents to target cytotoxic drugs to antigen-expressing tumour cells, whereupon they are internalized by said tumour cells, enabling the cytotoxic drug to take effect in specific tumour cells. This strategy has been further advanced through the conjugation of drugs to antibodies through cleavable linker molecules, enabling the ADC to be cleaved once internalised into a specific cell, releasing the cytotoxic drug such that it has greater activity.

In one embodiment, the present invention provides an antibody-drug conjugate comprising the antibody, or antigen-binding fragment thereof, disclosed herein.

In one embodiment, the present invention provides an antibody-drug conjugate comprising the antibody, or antigen-binding fragment thereof, disclosed herein and a coupling moiety.

In one embodiment, the present invention provides an antibody-drug conjugate comprising the antibody, or antigen-binding fragment thereof, disclosed herein and a coupling moiety connected by a cleavable linker-molecule.

In one embodiment, the present invention provides an antibody-drug conjugate comprising the antibody, or antigen-binding fragment thereof, disclosed herein and a coupling moiety connected by a non-cleavable linker-molecule.

In one embodiment, the present invention provides an antibody-drug conjugate comprising the antibody, or antigen-binding fragment thereof, disclosed herein and a coupling moiety wherein the coupling moiety is a drug.

In one embodiment, the present invention provides an antibody-drug conjugate comprising the antibody, or antigen-binding fragment thereof, disclosed herein and a coupling moiety wherein the coupling moiety is a toxin.

In one embodiment, the present invention provides an antibody-drug conjugate comprising the antibody, or antigen-binding fragment thereof, disclosed herein and a coupling moiety wherein the coupling moiety is a cytokine.

In one embodiment, the present invention provides an antibody-drug conjugate comprising the antibody, or antigen-binding fragment thereof, disclosed herein and a coupling moiety wherein the coupling moiety is an enzyme.

In one embodiment, the present invention provides an antibody-drug conjugate comprising the antibody, or antigen-binding fragment thereof, disclosed herein and a coupling moiety wherein the coupling moiety is a drug, toxin, cytokine, enzyme or a combination thereof.

### Antibody-Toxin Fusion Proteins

An antibody-toxin fusion protein, such as a recombinant immunotoxin, comprises an antibody, or antigen-binding fragment thereof, or immunoglobulin that is linked to a peptide toxin. Such an antibody-toxin fusion protein may be encoded by a recombinant DNA sequence that is subsequently expressed. The recombinant DNA sequence, also known as a fusion gene, may be expressed via an expression vector or plasmid that comprises the DNA segments which thus direct the synthesis of such a fusion protein. The antibody-toxin fusion protein, or recombinant immunotoxin, therefore comprises an amino acid sequence representing an antibody, antigen-binding fragment thereof, or immunoglobulin linked to peptide toxin.

In other words, a recombinant immunotoxin is a polypeptide in which a peptide toxin is genetically linked to an antibody component, often by a contiguous polypeptide linker.

Much like antibody-drug conjugates, antibody-toxin fusion proteins enable targeted accumulation and activation of the toxin for specific effect, promoting efficiency while minimising harm to normal cells.

In one embodiment, the present invention provides a recombinant fusion protein wherein the fusion protein comprises an antibody, or antigen-binding fragment thereof, disclosed herein that is linked to a peptide toxin.

In one embodiment, the present invention provides a recombinant fusion protein wherein the fusion protein comprises an antibody, or antigen-binding fragment thereof, as disclosed herein, wherein at least one of the heavy or light chain regions are linked to a peptide toxin.

In one embodiment, the present invention provides a recombinant fusion protein wherein the fusion protein comprises an antibody, or antigen-binding fragment thereof, disclosed herein, wherein at least one of SEQ ID NOs 14-20 and 29 are linked to a peptide toxin.

In one embodiment, the recombinant fusion protein comprises an antibody, or antigen-binding fragment thereof, disclosed herein, wherein at least one of SEQ ID NO 14 and SEQ

ID NO 16 are linked to a peptide toxin.

In one embodiment, the recombinant fusion protein comprises an antibody, or antigen-binding fragment thereof, disclosed herein, wherein at least one of SEQ ID NO 15 and SEQ ID NO 17 are linked to a peptide toxin.

In one embodiment, the recombinant fusion protein comprises an antibody, or antigen-binding fragment thereof, disclosed herein, wherein at least one of SEQ ID NO 14 and SEQ ID NO 18 are linked to a peptide toxin.

In one embodiment, the recombinant fusion protein comprises an antibody, or antigen-binding fragment thereof, disclosed herein, wherein at least one of SEQ ID NO 14 and SEQ ID NO 19 are linked to a peptide toxin.

In one embodiment, the recombinant fusion protein comprises an antibody, or antigen-binding fragment thereof, disclosed herein, wherein at least one of SEQ ID NO 14 and SEQ ID NO 20 are linked to a peptide toxin.

In one embodiment, the recombinant fusion protein comprises an antibody, or antigen-binding fragment thereof, disclosed herein, wherein at least one of SEQ ID NO 14 and SEQ

ID NO 17 are linked to a peptide toxin.

In one embodiment, the recombinant fusion protein comprises an antibody, or antigen-binding fragment thereof, disclosed herein, wherein at least one of SEQ ID NO 14 and SEQ ID NO 29 are linked to a peptide toxin.

### Nucleotide Sequences

The antibody, or antigen-binding fragment thereof, may be encoded by any suitable nucleotide sequence.

In some embodiments, the nucleotide sequence is codon-optimised, for example codon-optimised for expression in humans or plants. Cells may differ in their bias towards particular codons, some of which are used more. This codon bias corresponds to the relative abundance of particular tRNAs in a given cell type. As such, codons may be altered in a sequence such that they match with the tRNA with the greatest relative abundance in a cell type, thereby increasing expression. Similarly, by altering the sequence such that it matches the least abundant tRNA, one can reduce expression. Therefore, a greater degree of translational control is available. Codon usage tables are known in the art for a variety of organisms.

In one embodiment, the present invention provides a nucleic acid that encodes an antibody, or antigen-binding fragment thereof, as disclosed herein.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21.

In one embodiment, the nucleic acid encodes a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 23.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 23.

In one embodiment, the nucleic acid encodes a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 25.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 25.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 22.

In one embodiment, the nucleic acid encodes a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 24.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 22 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 24.

In one embodiment, the nucleic acid encodes a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 26.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 26.

In one embodiment, the nucleic acid encodes a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 27.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 27.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 24.

In one embodiment, the nucleic acid encodes a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 30.

In one embodiment, the nucleic acid encodes a light chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 21 and a heavy chain variable region having at least 70%, at least 75%, at least 80%, at least 85%, at least 90%, at least 91%, at least 92%, at least 93%, at least 94%, at least 95%, at least 96%, at least 97%, at least 98%, at least 99% or 100% identity to SEQ ID NO 30.

### Expression System

The present invention further relates to a method of production and engineering of antibodies, or antigen-binding fragments thereof, disclosed herein.

In some embodiments, a range of production hosts may be used for production of the antibody, or an antigen-binding fragment thereof, disclosed herein. Such hosts, which are utilized for the production of antibodies, or antigen-binding fragments thereof, may include, but are not limited to mammalian cells (eg. CHO cells), plant cells (eg. Nicotiana benthamiana cells), eukaryotic cells (eg. yeast cells) and microbial cells.

In some embodiments within the scope of the present disclosure and as known in the art, one or more plasmid expression vectors may be assembled and transfected into living cells for transient expression by the host. If multiple expression vectors are used, they can be transfected into the cells either separately or jointly, and expressed by the host. In a preferred embodiment, an engineered Nicotiana benthamiana (Nb) plant strain is the production host into which one or more expression vectors containing nucleic acids encoding the antibody, or antigen-binding fragments thereof, of the present disclosure are transfected.

In one embodiment, the plasmid expression vector is transfected into Agrobacterium hosts. In a preferred embodiment, the plasmid expression vector is transfected into Agrobacterium tumefaciens.

In a further embodiment, vector-carrying Agrobacterium are introduced into plant hosts by spray inoculation in the presence of mild abrasion. In a further embodiment, vector-carrying Agrobacterium are introduced into plant hosts by vacuum infiltration.

### FURTHER EMBODIMENTS

Monoclonal antibodies, including immunoglobulins, have been engineered and produced in various kinds of eukaryotic cells and other living cells. However, mammalian expression systems tend to be hampered by slow production turnaround cycles, and production through the use of certain cell lines may be constrained by the presence of mammalian viruses and other undesired compounds produced alongside the monoclonal antibodies in mammalian systems.

While not intending to limit the scope of the present embodiments, it is noted that plant based antibody production systems can avoid certain pitfalls of mammalian-based monoclonal antibody production. One such system, provided as a non-limiting example, utilizes an engineered Nicotiana benthamiana (Nb) plant strain expressing one or more expression vectors containing nucleic acid constructs for the formation of the desired mAbs. In some embodiments within the scope of the present disclosure, and as known in the art, one or more plasmid expression vectors are assembled and then transfected into living cells for transient expression by the host. If multiple expression vectors are used, they can be transfected into the cells, for example as found in plant tissue of Nb, either separately or jointly, and expressed by the host. An exemplary transient expression process uses Agrobacterium strains (e.g., Agrobacterium tumefaciens), in which the Agrobacterium are grown and infiltrated into whole plants, infecting the plant and introducing the genes to the plant cells. After infiltration and transient expression of the genes of interest in the germinated plants, proper feeding and care of the infiltrated plants provides for desired plant growth and ultimately harvesting of the mAb-containing infiltrated plant samples. The desired mAb product then is extracted from the harvested parts of the plant, generally followed by purification and characterization of the extracted monoclonal antibodies.

While the manner of introducing the vector-carrying Agrobacterium into these hosts is not intended as a limiting factor, spray inoculation in the presence of mild abrasion and vacuum infiltration are options. Again, other methods can be employed with plant hosts, and other non-plant hosts can be utilized with the nucleic acid constructs introduced to those hosts through known methods.

As will be appreciated by persons of ordinary skill in the relevant art, a range of other production hosts can be suitable for use in mAb production. Such hosts which are utilized for the production of mAbs in accordance with the present embodiments may include, but are not necessarily limited to, mammalian (e.g., CHO cells), plant (E.g., Nb plants or plant cells), yeast, and eukaryotic, and microbial cells. Through known steps available in the relevant literature, such hosts have been developed through genetic engineering, mutagenesis, or (in the case of plants) selective breeding aimed at enhancing mAb production or provide additional physicochemical characteristics in terms of increased or decreased molecular, metabolic, chemical, phenotypic, or other traits that affect protein formation by host. Present embodiments provide for representative amino acid sequences of monoclonal antibodies derived from murine sources, which have been humanized to modify the amino acid sequences to increase their similarity to antibody sequence variants found in humans. Thus, while not necessarily limited to those sequences specifically provided herein, amino acid sequences within the scope of this disclosure comprise humanized monoclonal antibodies, demonstrating anti-β-glucan activity or other activity favorable to neutralizing the effects of β-glucans. In some aspects, the anti-β-glucan activity is targeted to 1,3-β-glucan or 1,6-β-glucan molecules.

Such amino acid sequences may provide for humanized mAbs that take the form of a fully assembled, two-chain antibody; a single chain antibody; an antibody fragment comprising a variable region of an immunoglobulin antibody such as IgG, with antigen binding sites capable of binding to a molecule of interest and prompting an immunological response in an organism, including a mammal and particularly a human; an antibody fragment comprising one or more CDRs of a variable region from such an antibody; a human, chimeric, or humanized antibody; a multimeric antibody such as but not limited to a hexameric antibody; nanobodies, one or more individual immunoglobulin domains; a fusion protein comprising one or more antibodies or antibody portions of the present disclosure; and as antigen binding sequences of such antibodies, or as a combination of any of the above forms or other forms.

Still further, embodiments of the present disclosure include any products packaged in any number of forms (e.g., syringe, vial, blister packaging, inhaler). Such products may include appropriate buffers, additives, and excipients, and may be delivered in unit doses administered via syringe, pill, spray, inhaler, and other modalities through various routes that include, but are not limited to, subcutaneous, intramuscular, intradermal, injectable, oral, nasal, and topical administration or other forms, any of which may be as clinically indicated. Such products may be in the form of biologics or pharmaceutical compositions, such as having the antibody or antibodies of this disclosure in a clinically or therapeutically effective amount coupled to a suitable carrier, for example a non-infectious carrier such as viruses, virus-like particles, and nanoparticles.

Pharmaceutical compositions contemplated herein may include monoclonal antibodies as disclosed herein with a pharmaceutically acceptable carrier. Also, the scope of the present disclosure includes the mAbs themselves as disclosed herein, and methods for their production.

In view of the teachings herein, various novel mAbs which are produced according to the present embodiments as described and/or claimed will demonstrate several benefits, including improved affinity to specific antigens or target molecules, increased half-life, increased scalability for higher yields, and cost-effectiveness, to name some. Also, to accomplish these goals, there are described herein a number of exemplary antibody sequences which target β-glucans (i.e., β-glucan molecules, e.g., 1,3-β-glucan and 1,6-β-glucan molecules).

Present embodiments include one or more humanized monoclonal antibodies capable of binding, and which do bind, to β-glucan molecules. Five humanized heavy chain amino acid sequences are identified specifically, along with five humanized light chain amino acid sequences. Present embodiments include a number of humanized mAbs formed from combinations of the heavy and light chains.

Humanization and Purification of Subject mAbs

While various humanization techniques have been attempted, and many of which are published or otherwise are known in the art, a balance always exists between conserving the binding affinity of animal-derived antibody while achieving suitable compatibility of the humanized mAb when administered to a human subject. With regard to the inventive amino acid sequences disclosed herein, a humanization process started by performing sequence analysis of appropriate human acceptor frameworks on both heavy chain and light chain sequences, thereby resulting in identification of amino acid differences between the human acceptor frameworks and a known murine antibody whose binding activity to β-glucans can be determined.

Once amino acid differences were identified, rankings were made based on an extent of biochemical similarity between human subjects and murine subjects. From these steps, three variable heavy sequences were designed, along with three variable light sequences. The following table (Table 1) compares the identity of the human sequence to wt (i.e., murine wild type-derived) as a percentage, reflecting that the percentages were higher for all five heavy chain variants than for the wt control.

TABLE 1

| Identifier | GI of VH [%] |
|---|---|
| wt | 68.2 |
| VH_V1 | 77.7 |
| VH_V2 | 85.0 |
| VH_V3 | 90.7 |
| VH_V4 | 88.8 |
| VH_V5 | 92.5 |

By factoring the effects of surface-displayed amino acids and comparing possible variants to similar human antibody structures in the Clinical NSG (Next-Generation Sequencing Database), two additional variable heavy and two variable light sequences were designed. Each of these variants sought to have an increasing number of amino acid exchanges that were identical to the human acceptor framework, as well as an increasing number of conserved amino acids from the murine source as found in the antigen binding sites.

Additionally, data arrived at in the same way for the variable light chains and showing the same trends among the variants and the control is provided in Table 2.

TABLE 2

| Identifier | GI of VH [%] |
|---|---|
| wt | 82.5 |
| VL_V1 | 89.3 |
| VL_V2 | 91.3 |
| VL_V3 | 93.2 |
| VL_V4 | 92.2 |
| VL_V5 | 96.1 |

Accordingly, five heavy chain and five light chain sequences were identified, which were expressed in VH and VL as paired combinations and whose sequences are disclosed herein as SEQ ID NO: 14, SEQ ID NO: 17, SEQ ID NO: 29, and SED ID NO: 31-SEQ ID NO: 37. While not intending to limit these teachings to a single approach, each humanized mAb tested as discussed herein was produced by expressing the heavy chain sequence on a first expression vector, and the light chain sequence on a second expression vector. For non-limiting illustrative purposes, PVX (vector with a potato virus X backbone) and TVCV (vector with a tomato vein clearing virus backbone) are suitable vectors for these purposes. Once produced, monoclonal antibodies of the present disclosure may be purified by methods known in the relevant art. For example, U.S. Pat. No. 10,214,747 (“Method of purifying monoclonal antibodies,” issued Feb. 26, 2019), the contents of which are fully incorporated by reference herein, describes a monoclonal antibody purification platform that includes a procedure and processes to purify a wide array of different antibodies, which would be suitable for purifying antibodies produced in accordance with the present disclosure.

### Further Amino Acid Sequences According to Present Embodiments

The exemplary amino acid sequences described herein may be expressed in any of a number of optional ways, and in any combination of heavy and light amino acid chains. It will be appreciated that while exemplary sequences are provided, the scope of the present disclosure would also encompass other sequences including portions of the sequences identified, sequences presenting common structural features, and longer sequences in which the exemplary sequences provided here are included.

Sequences disclosed herein include SEQ ID NO: 14, SEQ ID NO: 17, SEQ ID NO: 29 and SEQ ID NOs: 31-SEQ ID NO: 37 (See Table 3 below.) SEQ ID NO: 17, SEQ ID NO: 29 and SEQ ID NO: 31-SEQ ID NO: 33 are heavy chain amino acid sequences. SEQ ID NO: 14 and SEQ ID NO: 34-SEQ ID NO: 37 are light chain amino acid sequences.

According to present embodiments, a heavy chain for a humanized monoclonal antibody comprises an amino acid sequence as contained in SEQ ID NO: 17. The amino acid sequence of SEQ ID NO: 17 may be combined with a light chain amino acid sequence to form a humanized antibody, wherein the light chain comprises an amino acid sequence contained in any of SEQ ID NO: 14 and SEQ ID NO: 34-SEQ ID NO: 37. As non-limiting examples, a combination of heavy and light chains may use SEQ ID NO: 17 combined with SEQ ID NO: 35 or SEQ ID NO: 17 combined with SEQ ID NO: 14.

Also, embodiments according to the present disclosure include those wherein a heavy chain for a humanized monoclonal antibody comprises an amino acid sequence as contained in SEQ ID NO: 31. The amino acid sequence of SEQ ID NO: 31 may be combined with a light chain amino acid sequence to form a humanized antibody, wherein the light chain comprises an amino acid sequence contained in any of SEQ ID NO: 14 and SEQ ID NO: 34-SEQ ID NO: 37. As non-limiting examples, a combination of heavy and light chains may use SEQ ID NO: 31 combined with SEQ ID NO: 35 or SEQ ID NO: 31 combined with SEQ ID NO: 14.

Embodiments according to the present disclosure also include those wherein a heavy chain for a humanized monoclonal antibody comprises an amino acid sequence as contained in SEQ ID NO: 32. The amino acid sequence of SEQ ID NO: 32 may be combined with a light chain amino acid sequence to form a humanized antibody, wherein the light chain comprises an amino acid sequence contained in any of SEQ ID NO: 14 and SEQ ID NO: 34-SEQ ID NO: 37. As non-limiting examples, a combination of heavy and light chains may use SEQ ID NO: 32 combined with SEQ ID NO: 35 or SEQ ID NO: 32 combined with SEQ ID NO: 14.

Still further, embodiments of the present disclosure include those wherein a heavy chain for a humanized monoclonal antibody comprises an amino acid sequence as contained in SEQ ID NO: 29. The amino acid sequence of SEQ ID NO: 29 may be combined with a light chain amino acid sequence to form the humanized antibody, wherein the light chain comprises an amino acid sequence contained in any of SEQ ID NO: 14 and SEQ ID NO: 34-SEQ ID NO: 37. As non-limiting examples, a combination of heavy and light chains may use SEQ ID NO: 29 combined with SEQ ID NO: 35 or SEQ ID NO: 29 combined with SEQ ID NO: 14.

In addition, embodiments of the present disclosure include those wherein a heavy chain for a humanized monoclonal antibody comprises an amino acid sequence as contained in SEQ ID NO: 33. The amino acid sequence of SEQ ID NO: 33 may be combined with a light chain amino acid sequence to form the humanized antibody, wherein the light chain comprises an amino acid sequence contained in any of SEQ ID NO: 14 and SEQ ID NO: 34-SEQ ID NO: 37. As non-limiting examples, a combination of heavy and light chains may use SEQ ID NO: 33 combined with SEQ ID NO: 35 or SEQ ID NO: 33 combined with SEQ ID NO: 14.

Also, embodiments of the present disclosure include those wherein a light chain for a humanized monoclonal antibody comprises an amino acid sequence as contained in SEQ ID NO: 35. The amino acid sequence of SEQ ID NO: 36 may be combined with a heavy chain amino acid sequence to form the humanized antibody, wherein the heavy chain comprises an amino acid sequence contained in any of SEQ ID NO: 17, SEQ ID NO: 29 and SEQ ID NO: 31-SEQ ID NO: 33.

In addition, embodiments of the present disclosure include those wherein a light chain for a humanized monoclonal antibody comprises an amino acid sequence as contained in SEQ ID NO: 14. The amino acid sequence of SEQ ID NO: 14 may be combined with a heavy chain amino acid sequence to form the humanized antibody, wherein the heavy chain comprises an amino acid sequence contained in any of SEQ ID NO: 17, SEQ ID NO: 29 and SEQ ID NO: 31-SEQ ID NO: 33.

Still further, embodiments of the present disclosure include those wherein a light chain for a humanized monoclonal antibody comprises an amino acid sequence as contained in any of SEQ ID NO: 34 and SEQ ID NO: 36-SEQ ID NO: 37. The amino acid sequence of any of

SEQ ID NO: 34 and SEQ ID NO: 36-SEQ ID NO: 37 may be combined with a heavy chain amino acid sequence to form the humanized antibody, wherein the heavy chain comprises an amino acid sequence contained in any of SEQ ID NO 17, SEQ ID NO: 29 and SEQ ID NO: 31-SEQ ID NO: 33.

In accordance with the sequences identified above, SEQ ID NO 17, SEQ ID NO: 29 and SEQ ID NO: 31-SEQ ID NO: 33 are variant heavy chain variable regions. Accordingly, Table 3 associates each heavy chain variable region variant (VH) to one of the above heavy chain SEQ ID NO's.

Also, in accordance the sequences identified above, SEQ ID NO: 14 and SEQ ID NO: 34-SEQ ID NO: 37 are variant light chain variable regions. Accordingly, Table 3 associates each light chain variant (VK) to one of the above light chain SEQ ID NOs. In view of the above, three clones are provided herein: 3.1, 14.1, and 32.3.

Clone 3.1 is a VH_wt combined with VL_wild type murine-derived mAb used as a control so none of the aforementioned sequences is included in this clone.

Clone 14.1 is additionally identified as VH_V1_VL_V5, and comprises a combination of SEQ ID NO: 17_SEQ ID NO: 14.

Clone 32.3 is additionally identified as VH_V4_VK_V5. It comprises a combination of SEQ ID NO: 29_SEQ ID NO: 14.

It will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways. Also, it is to be understood that words and phrases used herein are for the purpose of description and should not be regarded as limiting. The use herein of “including”, “comprising”, “e.g.”, “containing”, or “having” and variations of those words is meant to encompass the items listed thereafter, and equivalents of those, as well as additional items. Accordingly, the foregoing descriptions of several embodiments and alternatives are meant to illustrate, rather than to serve as limits on the scope of what has been disclosed herein.

The descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

### NUMBERED EMBODIMENTS

1. An antibody, or antigen-binding fragment thereof, wherein the antibody or antigen-binding fragment comprises a light chain variable region comprising:
(a) a CDRL1 of SEQ ID NO 1;
(b) a CDRL2 selected from the group consisting of SEQ ID NO 2 and SEQ ID NO 3; and
(c) a CDRL3 of SEQ ID NO 4;
and a heavy chain variable region comprising:
(d) a CDRH1 selected from the group consisting of SEQ ID NOs 5-7;
(e) a CDRH2 selected from the group consisting of SEQ ID NOs 8-12 and SEQ ID NO 28; and
(f) a CDRH3 of SEQ ID NO 13.

2. The antibody, or antigen-binding fragment thereof, of embodiment 1, wherein the light chain variable region comprises:
(a) a CDRL1 of SEQ ID NO 1;
(b) a CDRL2 of SEQ ID NO 2; and
(c) a CDRL3 of SEQ ID NO 4.

3. The antibody, or antigen-binding fragment thereof, of embodiment 1 or embodiment 2, wherein the light chain variable region comprises an amino acid sequence having at least 70% identity to SEQ ID NO 14.

4. The antibody, or antigen-binding fragment thereof, of any one of embodiments 1 to 3, wherein the heavy chain variable region comprises:
(a) a CDRH1 of SEQ ID NO 5;
(b) a CDRH2 of SEQ ID NO 8; and
(c) a CDRH3 of SEQ ID NO 13.

5. The antibody, or antigen-binding fragment thereof, of any one of embodiments 1 to 4, wherein the heavy chain variable region comprises an amino acid sequence having at least 70% identity to SEQ ID NO 16.

6. The antibody, or antigen-binding fragment thereof, of any one of embodiments 1 to 3, wherein the heavy chain variable region comprises:
(a) a CDRH1 of SEQ ID NO 5;
(b) a CDRH2 of SEQ ID NO 10; and
(c) a CDRH3 of SEQ ID NO 13.

7. The antibody, or antigen-binding fragment thereof, of any one of embodiments 1 to 3, or 6, wherein the heavy chain variable region comprises an amino acid sequence having at least 70% identity to SEQ ID NO 18.

8. The antibody, or antigen-binding fragment thereof, of embodiment 1, wherein the light chain variable region comprises:
(a) a CDRL1 of SEQ ID NO 1;
(b) a CDRL2 of SEQ ID NO 3; and
(c) a CDRL3 of SEQ ID NO 4.

9. The antibody, or antigen-binding fragment thereof, of embodiment 1 or embodiment 8, wherein the light chain variable region comprises an amino acid sequence having at least 70% identity to SEQ ID NO 15.

10. The antibody, or antigen-binding fragment thereof, of any one of embodiments 1 to 3, 8 or 9, wherein the heavy chain variable region comprises:
(a) a CDRH1 of SEQ ID NO 6;
(b) a CDRH2 of SEQ ID NO 9; and
(c) a CDRH3 of SEQ ID NO 13.

11. The antibody, or antigen-binding fragment thereof, of any one of embodiments 1 to 3, 8, 9, or 10, wherein the heavy chain variable region comprises an amino acid sequence having at least 70% identity to SEQ ID NO 17.

12. The antibody, or antigen-binding fragment thereof, of any one of embodiments 1 to 3 wherein the heavy chain variable region comprises:
(a) a CDRH1 of SEQ ID NO 5;
(b) a CDRH2 of SEQ ID NO 11; and
(c) a CDRH3 of SEQ ID NO 13.

13. The antibody, or antigen-binding fragment thereof, of any one of embodiments 1 to 3 or 12 wherein the heavy chain variable region comprises an amino acid sequence having at least 70% identity to SED ID NO 19.

14. The antibody, or antigen-binding fragment thereof, of any one of embodiments 1 to 3, wherein the heavy chain variable region comprises:
(a) a CDRH1 of SEQ ID NO 7;
(b) a CDRH2 of SEQ ID NO 12; and
(c) a CDRH3 of SEQ ID NO 13.

15. The antibody, or antigen-binding fragment thereof, of any one of embodiments 1 to 3 or 14, wherein the heavy chain variable region comprises an amino acid sequence having at least 70% identity to SEQ ID NO 20.

16. The antibody, or antigen-binding fragment thereof, of any one of embodiments 1 to 3, wherein the heavy chain variable region comprises:
(a) a CDRH1 of SEQ ID NO 6;
(b) a CDRH2 of SEQ ID NO 28; and
(c) a CDRH3 of SEQ ID NO 13.

17. The antibody, or antigen-binding fragment thereof, of any one of embodiments 1 to 3 or 16, wherein the heavy chain variable region comprises an amino acid sequence having at least 70% identity to SEQ ID NO 29.

18. An antibody, or antigen-binding fragment thereof, wherein the antibody, or antigen-binding fragment thereof, comprises a light chain variable region comprising an amino acid sequence having at least 70% identity to SEQ ID NO 14.

19. An antibody, or antigen-binding fragment thereof, wherein the antibody, or antigen-binding fragment thereof, comprises a heavy chain variable region comprising an amino acid sequence having at least 70% sequence identity to SEQ ID NO 16.

20. An antibody, or antigen-binding fragment thereof, wherein the antibody, or antigen binding fragment thereof, comprises a light chain variable region comprising an amino acid sequence having at least 70% sequence identity to SEQ ID NO 14 and wherein the antibody, or antigen binding fragment thereof, comprises a heavy chain variable region comprising an amino acid sequence having at least 70% sequence identity to SEQ ID NO 16.

21. An antibody, or antigen-binding fragment thereof, wherein the antibody, or antigen-binding fragment thereof, comprises a heavy chain variable region comprising an amino acid sequence having at least 70% sequence identity to SEQ ID NO 18.

22. An antibody, or antigen-binding fragment thereof, wherein the antibody, or antigen binding fragment thereof, comprises a light chain variable region comprising an amino acid sequence having at least 70% sequence identity to SEQ ID NO 14 and wherein the antibody, or antigen binding fragment thereof, comprises a heavy chain variable region comprising an amino acid sequence having at least 70% sequence identity to SEQ ID NO 18.

23. An antibody, or antigen-binding fragment thereof, wherein the antibody, or antigen-binding fragment thereof, comprises a light chain variable region comprising an amino acid sequence having at least 70% identity to SEQ ID NO 15.

24. An antibody, or antigen-binding fragment thereof, wherein the antibody, or antigen-binding fragment thereof, comprises a heavy chain variable region comprising an amino acid sequence having at least 70% sequence identity to SEQ ID NO 17.

25. An antibody, or antigen-binding fragment thereof, wherein the antibody, or antigen binding fragment thereof, comprises a light chain variable region comprising an amino acid sequence having at least 70% sequence identity to SEQ ID NO 15 and wherein the antibody, or antigen binding fragment thereof, comprises a heavy chain variable region comprising an amino acid sequence having at least 70% sequence identity to SEQ ID NO 17.

26. An antibody, or antigen-binding fragment thereof, wherein the antibody, or antigen binding fragment thereof, comprises a light chain variable region comprising an amino acid sequence having at least 70% sequence identity to SEQ ID NO 14 and wherein the antibody, or antigen binding fragment thereof, comprises a heavy chain variable region comprising an amino acid sequence having at least 70% sequence identity to SEQ ID NO 17.

27. An antibody, or antigen-binding fragment thereof, wherein the antibody, or antigen-binding fragment thereof, comprises a heavy chain variable region comprising an amino acid sequence having at least 70% sequence identity to SEQ ID NO 19.

28. An antibody, or antigen-binding fragment thereof, wherein the antibody, or antigen-binding fragment thereof, comprises a light chain variable region comprising an amino acid sequence having at least 70% sequence identity to SEQ ID NO 14 and wherein the antibody, or antigen-binding fragment thereof, comprises a heavy chain variable region comprising an amino acid sequence having at least 70% sequence identity to SEQ ID NO 19.

29. An antibody, or antigen-binding fragment thereof, wherein the antibody, or antigen-binding fragment thereof, comprises a heavy chain variable region comprising an amino acid sequence having at least 70% sequence identity to SEQ ID NO 20.

30. An antibody, or antigen-binding fragment thereof, wherein the antibody, or antigen-binding fragment thereof, comprises a light chain variable region comprising an amino acid sequence having at least 70% sequence identity to SEQ ID NO 14 and wherein the antibody, or antigen-binding fragment thereof, comprises a heavy chain variable region comprising an amino acid sequence having at least 70% sequence identity to SEQ ID NO 20.

31. An antibody, or antigen-binding fragment thereof, wherein the antibody, or antigen-binding fragment thereof, comprises a heavy chain variable region comprising an amino acid sequence having at least 70% sequence identity to SEQ ID NO 29.

32. An antibody, or antigen-binding fragment thereof, wherein the antibody, or antigen-binding fragment thereof, comprises a light chain variable region comprising an amino acid sequence having at least 70% sequence identity to SEQ ID NO 14 and wherein the antibody, or antigen-binding fragment thereof, comprises a heavy chain variable region comprising an amino acid sequence having at least 70% sequence identity to SEQ ID NO 29.

33. The antibody, or antigen-binding fragment thereof, of any one of embodiments 3, 5, 7, 9, 11, 13, 15, and 17 to 32 wherein the antibody, or antigen-binding fragment thereof, has at least 75%, at least 80%, at least 85%, at least 90%, at least 95%, at least 99%, or 100% sequence identity to the recited sequence.

34. The antibody, or antigen-binding fragment thereof, of any one of the preceding embodiments, wherein the antibody or antigen binding fragment is a single-chain variable fragment (scFV), a single-domain antibody (sdAb), an antigen-binding fragment (Fab), or a fragment antibody (F(ab′)2).

35. The antibody, or antigen-binding fragment thereof, of any one of the preceding embodiments, wherein the antibody or antigen-binding fragment is IgG, IgA, IgM, IgE or IgD isotype or any allotype thereof.

36. The antibody, or antigen-binding fragment thereof, of embodiment 35, wherein the antibody or antigen-binding fragment is IgG1 isotype or any allotype thereof.

37. The antibody, or antigen-binding fragment thereof, of embodiment 35, wherein the antibody or antigen-binding fragment is IgG2 isotype or any allotype thereof.

38. The antibody, or antigen-binding fragment thereof, of embodiment 35, wherein the antibody or antigen-binding fragment is IgG3 isotype or any allotype thereof.

39. The antibody, or antigen-binding fragment thereof, of embodiment 35, wherein the antibody or antigen-binding fragment is IgG4 isotype or any allotype thereof.

40. The antibody, or antigen-binding fragment thereof, of embodiment 35, wherein the antibody or antigen-binding fragment is IgA1 isotype or any allotype thereof.

41. The antibody, or antigen-binding fragment thereof, of embodiment 35, wherein the antibody or antigen-binding fragment is IgA2 isotype or any allotype thereof.

42. The antibody, or antigen-binding fragment thereof, of embodiment 35, wherein the antibody or antigen-binding fragment is IgD1 isotype or any allotype thereof.

43. The antibody, or antigen-binding fragment thereof, of embodiment 35, wherein the antibody or antigen-binding fragment is IgD2 isotype or any allotype thereof.

44. The antibody, or antigen-binding fragment thereof, of any one of the preceding embodiments, wherein the antibody or antigen-binding fragment is humanized.

45. The antibody, or antigen-binding fragment thereof, of any one of the preceding embodiments, wherein the antibody binds to a β-glucan.

46. The antibody, or antigen-binding fragment thereof, of embodiment 45, wherein the β-glucan is a 1,3-β-glucan or a 1,6-β-glucan.

47. The antibody, or antigen-binding fragment thereof, of embodiment 45 or embodiment 46, wherein the antibody binds to laminarin.

48. The antibody, or antigen-binding fragment thereof, of any one of the preceding embodiments, for use in a bispecific antigen-binding protein, antibody, or antigen-binding fragment thereof, that binds to two different antigens or epitopes.

49. The antibody, or antigen-binding fragment thereof, of any one of the preceding embodiments, for use in a multi-specific antigen-binding protein, antibody, or antigen-binding fragment thereof, that binds to two or more different antigens or epitopes.

50. The antibody, or antigen-binding fragment thereof, of any one of the preceding embodiments, wherein the antibody, or antigen-binding fragment thereof, is in the form of a homo-dimer or homo-multimer.

51. The antibody, or antigen-binding fragment thereof, of any one of the preceding embodiments, wherein the antibody, or antigen-binding fragment thereof, have an EC50 less than 0.2 μg/mL.

52. The antibody, or antigen-binding fragment thereof, of embodiment 51, wherein the antibody, or antigen-binding fragment thereof, have an EC50 less than 0.1 μg/mL.

53. The antibody, or antigen-binding fragment thereof, of any one of the preceding embodiments, wherein the antibody or antigen-binding fragment has a Kp value lower than 1.5×10⁻⁸ M.

54. The antibody, or antigen-binding fragment thereof, of embodiment 53, wherein the antibody or antigen-binding fragment has a Kp value lower than 1×10⁻⁸ M.

55. The antibody, or antigen-binding fragment thereof, of embodiment 54, wherein the antibody or antigen-binding fragment has a Kp value lower than 5×10⁻⁹ M.

56. The antibody, or antigen-binding fragment thereof, of any one of the preceding embodiments, wherein the antibody or antigen-binding fragment is thermostable up to 80° C.

57. The antibody, or antigen-binding fragment thereof, of embodiment 56, wherein the antibody or antigen binding fragment is thermostable up to 70° C.

58. The antibody, or antigen-binding fragment thereof, of embodiment 57, wherein the antibody or antigen-binding fragment is thermostable up to 60° C.

59. The antibody, or antigen-binding fragment thereof, of any one of the preceding embodiments, for use as an anti-fungal agent.

60. The antibody, or antigen-binding fragment thereof, of any one of the preceding embodiments, for use in the treatment of fungal infection.

61. The antibody, or antigen-binding fragment thereof, of any one of the preceding embodiments, for use in the manufacture of a medicament for the treatment of fungal infection.

62. A method of treating fungal infection comprising administering the antibody, or antigen-binding fragment thereof, of any one of the preceding embodiments.

63. The antibody, or antigen-binding fragment thereof, for use according to embodiments 59-61 or the method of embodiment 62, wherein the fungal infection is an Aspergillus infection.

64. The antibody, or antigen-binding fragment thereof, or the method, of embodiment 63 wherein the Aspergillus infection is an Aspergillus fumigatus infection.

65. The antibody, or antigen-binding fragment thereof, for use according to embodiment 59-61 or the method of embodiment 62, wherein the fungal infection is a Candida infection.

66. The antibody, or antigen-binding fragment thereof, or the method, of embodiment 65 wherein the Candida infection is a Candida albicans or Candida auris infection.

67. A recombinant fusion protein wherein the fusion protein comprises an antibody, or antigen-binding fragment thereof, of any of the preceding embodiments, and a peptide toxin.

68. The recombinant fusion protein of embodiment 67 wherein at least one of SEQ ID NO 14 and SEQ ID NO 16 are linked to the peptide toxin.

69. The recombinant fusion protein of embodiment 67 wherein at least one of SEQ ID NO 15 and SEQ ID NO 17 are linked to the peptide toxin.

70. The recombinant fusion protein of embodiment 67 wherein at least one of SEQ ID NO 14 and SEQ ID NO 18 are linked to the peptide toxin.

71. The recombinant fusion protein of embodiment 67 wherein at least one of SEQ ID NO 14 and SEQ ID NO 19 are linked to the peptide toxin.

72. The recombinant fusion protein of embodiment 67 wherein at least one of SEQ ID NO 14 and SEQ ID NO 20 are linked to the peptide toxin.

73. The recombinant fusion protein of embodiment 67 wherein at least one of SEQ ID NO 14 and SEQ ID NO 17 are linked to the peptide toxin.

74. The recombinant fusion protein of embodiment 67 wherein at least one of SEQ ID NO 14 and SEQ ID NO 29 are linked to the peptide toxin.

75. The antibody, or antigen-binding fragment thereof, of any one of the preceding embodiments wherein the antibody, or antigen binding fragment thereof, is conjugated to a coupling moiety.

76. The antibody, or antigen-binding fragment thereof, of embodiment 75 wherein the coupling moiety is a drug.

77. The antibody, or antigen-binding fragment thereof, of embodiment 75 wherein the coupling moiety is a toxin.

78. The antibody, or antigen-binding fragment thereof, of embodiment 75 wherein the coupling moiety is a cytokine.

79. The antibody, or antigen-binding fragment thereof, of embodiment 75 wherein the coupling moiety is an enzyme.

80. The antibody, or antigen-binding fragment thereof, of any one of embodiments 75-79, wherein the antibody, or antigen-binding fragment thereof, and the coupling moiety are conjugated by a linker molecule.

81. The antibody, or antigen-binding fragment thereof, of embodiment 80 wherein the linker molecule is a cleavable linker molecule.

82. A nucleic acid encoding an antibody, or an antigen-binding fragment thereof, according to any one of the preceding embodiments.

83. A nucleic acid encoding a light chain variable region having at least 70% sequence identity to SEQ ID NO 21.

84. A nucleic acid encoding a heavy chain variable region having at least 70% sequence identity to SEQ ID NO 23.

85. A nucleic acid encoding a light chain variable region having at least 70% sequence identity to SEQ ID NO 21, and a heavy chain variable region having at least 70% sequence identity to SEQ ID NO 23.

86. A nucleic acid encoding a light chain variable region having at least 70% sequence identity to SEQ ID NO 22.

87. A nucleic acid encoding a heavy chain variable region having at least 70% sequence identity to SEQ ID NO 24.

88. A nucleic acid encoding a light chain variable region having at least 70% sequence identity to SEQ ID NO 22, and a heavy chain variable region having at least 70% sequence identity to SEQ ID NO 24.

89. A nucleic acid encoding a heavy chain variable region having at least 70% sequence identity to SEQ ID NO 25.

90. A nucleic acid encoding a light chain variable region having at least 70% sequence identity to SEQ ID NO 21, and a heavy chain variable region having at least 70% sequence identity to SEQ ID NO 25.

91. A nucleic acid encoding a heavy chain variable region having at least 70% sequence identity to SEQ ID NO 26.

92. A nucleic acid encoding a light chain variable region having at least 70% sequence identity to SEQ ID NO 21, and a heavy chain variable region having at least 70% sequence identity to SEQ ID NO 26.

93. A nucleic acid encoding a heavy chain variable region having at least 70% sequence identity to SEQ ID NO 27.

94. A nucleic acid encoding a light chain variable region having at least 70% sequence identity to SEQ ID NO 21, and a heavy chain variable region having at least 70% sequence identity to SEQ ID NO 27.

95. A nucleic acid encoding a light chain variable region having at least 70% sequence identity to SEQ ID NO 21, and a heavy chain variable region having at least 70% sequence identity to SEQ ID NO 24.

96. A nucleic acid encoding a heavy chain variable region having at least 70% sequence identity to SEQ ID NO 30.

97. A nucleic acid encoding a light chain variable region having at least 70% sequence identity to SEQ ID NO 21, and a heavy chain variable region having at least 70% sequence identity to SEQ ID NO 30

98. The nucleic acid of any one of embodiments 83-97, wherein the nucleic acid has at least 75%, at least 80%, at least 85%, at least 90%, at least 95%, at least 99%, or 100% sequence identity to the recited sequence.

99. A compound which is an anti-β-glucan antibody or antigen-binding fragment thereof, wherein the antibody or antigen-binding fragment comprises a heavy chain variable region (VH) and a light chain variable region (VL), wherein a VH amino acid sequence is chosen from any one of SEQ ID NO: 29 and SEQ ID NO: 31-SEQ ID NO: 34 and a VL amino acid sequence is chosen from any one of SEQ ID NO: 14 and SEQ ID NO: 35-SEQ ID NO: 38.

100. The compound of embodiment 99, wherein the VH amino acid sequence is either SEQ ID NO: 31 or SEQ NO: 29 and the VL amino acid sequence is SEQ ID NO: 14.

TABLE 3
Summary of antibody sequences

| SEQ | | |
|---|---|---|
| ID No | Description | Sequence |
| 1 | CDRL1 of clone 39.1 | RSSQSLVYSNGNTHLN |
| | (KB14A.39.1), clone 40.1 | |
| | (KB14A.40.1), clone 41.2 | |
| | (KB14A.41.2), clone 42.1 | |
| | (KB14A.42.1), clone 43.1 | |
| | (KB14A.43.1), clone 14.1 | |
| | (KB14A.14.1) & clone 32.3 | |
| | (KB14A.32.3) | |
| 2 | CDRL2 of clone 39.1 | LVSNRDS |
| | (KB14A.39.1), clone 41.2 | |
| | (KB14A.41.2), clone 42.1 | |
| | (KB14A.42.1), clone 43.1 | |
| | (KB14A.43.1), clone 14.1 | |
| | (KB14A.14.1) & clone 32.3 | |
| | (KB14A.32.3) | |
| 3 | CDRL2 of clone 40.1 | LVSVRDS |
| | (KB14A.40.1) | |
| 4 | CDRL3 of clone 39.1 | VQGTHFPYT |
| | (KB14A.39.1), clone 40.1 | |
| | (KB14A.40.1), clone 41.2 | |
| | (KB14A.41.2), clone 42.1 | |
| | (KB14A.42.1), clone 43.1 | |
| | (KB14A.43.1), clone 14.1 | |
| | (KB14A.14.1) & clone 32.3 | |
| | (KB14A.32.3) | |
| 5 | CDRH1 of clone 39.1 | NSYWLE |
| | (KB14A.39.1), clone 41.2 | |
| | (KB14A.41.2) & clone 42.1 | |
| | (KB14A.42.1) | |
| 6 | CDRH1 of clone 40.1 | SYWLE |
| | (KB14A.40.1), clone 14.1 | |
| | (KB14A.14.1) & clone 32.3 | |
| | (KB14A.32.3) | |
| 7 | CDRH1 of clone 43.1 | IRYWLE |
| | (KB14A.43.1) | |
| 8 | CDRH2 of clone 39.1 | EILPGIGSTNYNQKFKG |
| | (KB14A.39.1) | |
| 9 | CDRH2 of clone 40.1 | EILPGSGSTNYNQKFKG |
| | (KB14A.40.1) & clone 14.1 | |
| | (KB14A.14.1) | |
| 10 | CDRH2 of clone 41.2 | EILPGSGGTIYNQKFKG |
| | (KB14A.41.2) | |
| 11 | CDRH2 of clone 42.1 | EILPGTGSTSYNQKFKG |
| | (KB14A.42.1) | |
| 12 | CDRH2 of clone 43.1 | EILPGSGGTNYNQKFKG |
| | (KB14A.43.1) | |
| 13 | CDRH3 of clone 39.1 | EGWYFDVW |
| | (KB14A.39.1), clone 40.1 | |
| | (KB14A.40.1), clone 41.2 | |
| | (KB14A.41.2), clone 42.1 | |
| | (KB14A.42.1) & clone 43.1 | |
| | (KB14A.43.1), clone 14.1 | |
| | (KB14A.14.1) & clone 32.3 | |
| | (KB14A.32.3) | |
| 14 | Light chain variable region of | DVVMTQSPLSLPVTLGQPASISCRSSQSLVYSN |
| | clone 39.1 (KB14A.39.1), | GNTHLNWLQQRPGQSPRRLIYLVSNRDSGVPD |
| | clone 41.2 (KB14A.41.2), | RFSGSGSGTDFTLKISRVEAEDVGVYYCVQGT |
| | clone 42.1 (KB14A.42.1), | HFPYTFGQGTKLEIK |
| | clone 43.1 (KB14A.43.1), | |
| | clone 14.1 (KB14A.14.1) & | |
| | clone 32.3 (KB14A.32.3) | |
| | [VK_V5-light chain variant 5] | |
| 15 | Light chain variable region of | DVVMTQSPLSLPVTLGQPASISCRSSQSLVYSN |
| | clone 40.1 (KB14A.40.1) | GNTHLNWLQQRPGQSPRRLIYLVSVRDSGVPD |
| | | RFSGSGSGTDFTLKISRVEAEDVGVYYCVQGT |
| | | HFPYTFGQGTKLEIK |
| 16 | Heavy chain variable region | QVQLQQSGAEVMKPGASVKVSCKASGYTLNSY |
| | of clone 39.1 (KB14A.39.1) | WLEWVRQRPGHGLEWIGEILPGIGSTNYNQKF |
| | | KGRVTFTADTSTNTAYMELSSLTSEDTAVYYCA |
| | | REGWYFDVWGAGTTVTVSS |
| 17 | Heavy chain variable region | QVQLQQSGAEVMKPGASVKVSCKASGYTLSSY |
| | of clone 40.1 (KB14A.40.1) & | WLEWVRQRPGHGLEWIGEILPGSGSTNYNQKF |
| | clone 14.1 (KB14A.14.1) | KGRVTFTADTSTNTAYMELSSLTSEDTAVYYCA |
| | [VH_V1-heavy chain variant | REGWYFDVWGAGTTVTVSS |
| | 1] | |
| 18 | Heavy chain variable region | QVQLQQSGAEVMKPGASVKVSCKASGYTLNSY |
| | of clone 41.2 (KB14A.41.2) | WLEWVRQRPGHGLEWIGEILPGSGGTIYNQKF |
| | | KGRVTFTADTSTNTAYMELSSLTSEDTAVYYCA |
| | | REGWYFDVWGAGTTVTVSS |
| 19 | Heavy chain variable region | QVQLQQSGAEVMKPGASVKVSCKASGYTLNSY |
| | of clone 42.1 (KB14A.42.1) | WLEWVRQRPGHGLEWIGEILPGTGSTSYNQKF |
| | | KGRVTFTADTSTNTAYMELSSLTSEDTAVYYCA |
| | | REGWYFDVWGAGTTVTVSS |
| 20 | Heavy chain variable region | QVQLQQSGAEVMKPGASVKVSCKASGYTLIRY |
| | of clone 43.1 (KB14A.43.1) | WLEWVRQRPGHGLEWIGEILPGSGGTNYNQKF |
| | | KGRVTFTADTSTNTAYMELSSLTSEDTAVYYCA |
| | | REGWYFDVWGAGTTVTVSS |
| 21 | Nucleic acid sequence | GATGTTGTGATGACTCAGTCTCCTCTGAGCCT |
| | encoding light chain variable | TCCTGTTACTCTTGGTCAGCCTGCTAGCATCT |
| | region of clone 39.1 | CTTGCAGGTCATCTCAGTCTCTGGTGTACAGC |
| | (KB14A.39.1), clone 41.2 | AATGGTAACACCCACCTTAACTGGCTTCAACA |
| | (KB14A.41.2), clone 42.1 | GAGGCCTGGTCAATCTCCTAGAAGGCTTATCT |
| | (KB14A.42.1), clone 43.1 | ACCTGGTGAGCAACAGAGACTCTGGTGTGCC |
| | (KB14A.43.1), clone 14.1 | TGATAGGTTCAGCGGTTCTGGTTCTGGTACTG |
| | (KB14A.14.1) & clone 32.3 | ACTTCACCCTGAAGATCAGCAGAGTTGAGGC |
| | (KB14A.32.3) | TGAGGATGTGGGAGTTTACTACTGCGTTCAG |
| | | GGTACTCACTTCCCTTACACTTTCGGTCAGGG |
| | | CACCAAGCTTGAGATTAAG |
| 22 | Nucleic acid sequence | GATGTTGTGATGACTCAGTCTCCTCTGAGCCT |
| | encoding light chain variable | TCCTGTTACTCTTGGTCAGCCTGCTAGCATCT |
| | region of clone 40.1 | CTTGCAGGTCATCTCAGTCTCTGGTGTACAGC |
| | (KB14A.40.1) | AATGGTAACACCCACCTTAACTGGCTTCAACA |
| | | GAGGCCTGGTCAATCTCCTAGAAGGCTTATCT |
| | | ACCTGGTGAGCGTGAGAGATTCTGGTGTGCC |
| | | TGATAGGTTCAGCGGTTCTGGTTCTGGTACTG |
| | | ACTTCACCCTGAAGATCAGCAGAGTTGAGGC |
| | | TGAGGATGTGGGAGTTTACTACTGCGTTCAG |
| | | GGTACTCACTTCCCTTACACTTTCGGTCAGGG |
| | | CACCAAGCTTGAGATTAAG |
| 23 | Nucleic acid sequence | CAGGTTCAACTTCAACAGTCAGGCGCTGAGG |
| | encoding heavy chain | TTATGAAGCCTGGTGCTTCTGTGAAAGTGAGC |
| | variable region of clone 39.1 | TGCAAGGCTTCTGGTTACACCCTGAACTCTTA |
| | (KB14A.39.1) | CTGGCTTGAGTGGGTTAGACAGAGGCCTGGT |
| | | CATGGTCTTGAATGGATTGGTGAGATCCTGC |
| | | CTGGTATCGGCAGCACTAATTACAACCAGAA |
| | | GTTCAAGGGCCGAGTCACCTTCACTGCTGAT |
| | | ACTTCTACTAACACCGCCTACATGGAACTGTC |
| | | CAGCCTTACTTCTGAGGACACCGCTGTTTACT |
| | | ACTGCGCTAGAGAAGGCTGGTACTTCGATGT |
| | | TTGGGGTGCTGGTACTACCGTGACCGTTTCTT |
| | | CT |
| 24 | Nucleic acid sequence | CAGGTTCAACTTCAACAGTCAGGCGCTGAGG |
| | encoding heavy chain | TTATGAAGCCTGGTGCTTCTGTGAAAGTGAGC |
| | variable region of clone 40.1 | TGCAAGGCTAGCGGTTACACCCTGTCATCTTA |
| | (KB14A.40.1) & clone 14.1 | TTGGCTTGAGTGGGTGAGACAGAGGCCTGGT |
| | (KB14A.14.1) | CATGGTCTTGAATGGATTGGTGAGATCCTGC |
| | | CTGGTAGCGGTAGCACTAATTACAACCAGAA |
| | | GTTCAAGGGCCGAGTCACCTTCACTGCTGAT |
| | | ACTTCTACTAACACCGCCTACATGGAACTGTC |
| | | CAGCCTTACTTCTGAGGACACCGCTGTTTACT |
| | | ACTGCGCTAGAGAAGGCTGGTACTTCGATGT |
| | | TTGGGGTGCTGGTACTACCGTGACCGTTTCTT |
| | | CT |
| 25 | Nucleic acid sequence | CAGGTTCAACTTCAACAGTCAGGCGCTGAGG |
| | encoding heavy chain | TTATGAAGCCTGGTGCTTCTGTGAAAGTGAGC |
| | variable region of clone 41.2 | TGCAAGGCTTCTGGTTACACCCTGAACTCTTA |
| | (KB14A.41.2) | CTGGCTTGAGTGGGTTAGACAGAGGCCTGGT |
| | | CATGGTCTTGAATGGATTGGTGAGATCCTGC |
| | | CTGGTTCTGGTGGCACTATCTACAACCAGAA |
| | | GTTCAAGGGCCGAGTCACCTTCACTGCTGAT |
| | | ACTTCTACTAACACCGCCTACATGGAACTGTC |
| | | CAGCCTTACTTCTGAGGACACCGCTGTTTACT |
| | | ACTGCGCTAGAGAAGGCTGGTACTTCGATGT |
| | | TTGGGGTGCTGGTACTACCGTGACCGTTTCTT |
| | | CT |
| 26 | Nucleic acid sequence | CAGGTTCAACTTCAACAGTCAGGCGCTGAGG |
| | encoding heavy chain | TTATGAAGCCTGGTGCTTCTGTGAAAGTGAGC |
| | variable region of clone 42.1 | TGCAAGGCTTCTGGTTACACCCTGAACTOTTA |
| | (KB14A.42.1) | CTGGCTTGAGTGGGTTAGACAGAGGCCTGGT |
| | | CATGGTCTTGAATGGATTGGTGAGATTCTGCC |
| | | TGGTACTGGCAGCACCTCTTACAACCAGAAG |
| | | TTCAAGGGCCGAGTTACCTTCACCGCTGATA |
| | | CTTCTACTAACACCGCCTACATGGAACTGTCC |
| | | AGCCTTACTTCTGAGGACACCGCTGTTTACTA |
| | | CTGCGCTAGAGAAGGCTGGTACTTCGATGTT |
| | | TGGGGTGCTGGTACTACCGTGACCGTTTCTT |
| | | CT |
| 27 | Nucleic acid sequence | CAGGTTCAACTTCAACAGTCAGGCGCTGAGG |
| | encoding heavy chain | TTATGAAGCCTGGTGCTTCTGTGAAAGTGAGC |
| | variable region of clone 43.1 | TGCAAGGCTTCTGGTTACACCCTCATTAGGTA |
| | (KB14A.43.1) | CTGGCTTGAGTGGGTTAGACAGAGGCCTGGT |
| | | CATGGTCTTGAATGGATTGGTGAGATCCTGC |
| | | CTGGTAGCGGTGGCACTAATTACAACCAGAA |
| | | GTTCAAGGGCCGAGTCACCTTCACTGCTGAT |
| | | ACTTCTACTAACACCGCCTACATGGAACTGTC |
| | | CAGCCTTACTTCTGAGGACACCGCTGTTTACT |
| | | ACTGCGCTAGAGAAGGCTGGTACTTCGATGT |
| | | TTGGGGTGCTGGTACTACCGTGACCGTTTCTT |
| | | CT |
| 28 | CDRH2 of clone 32.3 | EILPGSGSTNYAQKFQG |
| | (KB14A.32.3) | |
| 29 | Heavy chain variable region | QVQLVQSGAEVKKPGSSVKVSCKASGYTLSSY |
| | of clone 32.3 (KB14A.32.3) | WLEWVRQAPGQGLEWIGEILPGSGSTNYAQKF |
| | [VH_V4-heavy chain variant | QGRVTITADESTSTAYMELSSLRSEDTAVYYCA |
| | 4] | REGWYFDVWGQGTTVTVSS |
| 30 | Nucleic acid sequence | CAGGTTCAGCTTGTTCAGTCAGGTGCTGAGG |
| | encoding heavy chain | TTAAGAAGCCGGGCTCATCTGTCAAAGTGAG |
| | variable region of clone 32.3 | CTGTAAGGCTAGCGGCTACACCCTGTCATCTT |
| | (KB14A.32.3) | ATTGGCTTGAGTGGGTGAGACAAGCTCCTGG |
| | | TCAAGGTCTTGAATGGATCGGTGAGATTCTGC |
| | | CTGGTAGCGGTTCTACTAACTACGCCCAAAA |
| | | GTTCCAGGGCAGAGTGACCATTACTGCTGAC |
| | | GAGTCTACTAGCACCGCCTACATGGAACTTA |
| | | GCAGCCTTAGGTCTGAGGACACCGCTGTTTA |
| | | TTACTGCGCTAGAGAAGGCTGGTACTTCGAT |
| | | GTTTGGGGTCAGGGTACTACCGTGACCGTTT |
| | | CTTCT |
| 31 | VH_V2-heavy chain variant | QVQLVQSGAEVKKPGSSVKVSCKASGGTFSSY |
| | 2 | WLEWVRQRPGQGLEWIGEILPGSGSTNYNQKF |
| | | QGRVTITADTSTNTAYMELSSLTSEDTAVYYCA |
| | | REGWYFDVWGAGTTVTVSS |
| 32 | VH_V3-heavy chain variant | QVQLVQSGAEVKKPGSSVKVSCKASGGTFSSY |
| | 3 | WLEWVRQAPGQGLEWIGEILPGSGSTNYAQKF |
| | | QGRVTITADESTSTAYMELSSLRSEDTAVYYCA |
| | | REGWYFDVWGQGTTVTVSS |
| 33 | VH_V5-heavy chain variant | QVQLVQSGAEVKKPGSSVKVSCKASGYTLSSY |
| | 5 | WIEWVRQAPGQGLEWMGEILPGSGTANYAQK |
| | | FQGRVTITADESTSTAYMELS |
| 34 | VK_V1-light chain variant 1 | DVVMTQSPLSLSVTLGQPASISCRSSQSLLYSN |
| | | GNTHLNWLLQRPGQSPRRLIYLVSKLDSGVPD |
| | | RFSGSGSGTDFTLKISRVEAEDVGFYYCVQGTH |
| | | FPYTFGGGTKLEIK |
| 35 | VK_V2-light chain variant 2 | DVVMTQSPLSLPVTLGQPASISCRSSQSLLYSN |
| | | GNTHLNWLLQRPGQSPRRLIYLVSKLDSGVPD |
| | | RFSGSGSGTDFTLKISRVEAEDVGVYYCVQGT |
| | | HFPYTFGGGTKLEIK |
| 36 | VK_V3-light chain variant 3 | DVVMTQSPLSLPVTLGQPASISCRSSQSLLYSN |
| | | GNTHLNWLQQRPGQSPRRLIYLVSKLDSGVPD |
| | | RFSGSGSGTDFTLKISRVEAEDVGVYYCVQGT |
| | | HFPYTFGQGTKLEIK |
| 37 | VK_V4-light chain variant 4 | DVVMTQSPLSLPVTLGQPASISCRSSQSLLYSN |
| | | GNTHLNWLLQRPGQSPRRLIYLVSKLDSGVPD |
| | | RFSGSGSGTDFTLKISRVEAEDVGVYYCVQGT |
| | | HFPYTFGQGTKLEIK |

### EXAMPLES

The invention will now be further described by way of examples, which are meant to serve to assist one of ordinary skill in the art in carrying out the invention and are not intended in any way to limit the scope of the invention.

The antibodies tested in the following examples were >99% pure following a two-step chromatography purification, as indicated by the column HPLC SEC Final % Monomer as indicated in Table 4.

TABLE 4

| Test Article | HPLC SEC Final % Monomer |
|---|---|
| Clone 39 | 100 |
| Clone 40 | 100 |
| Clone 41 | 100 |
| Clone 42 | 100 |
| Clone 43 | 100 |
| Clone 14 | 100 |
| Clone 32 | 99.65 |

### Example 1—Laminarin Binding (ELISA) to Determine EC50 Values of Initial Humanized and Next Generation Affinity Matured Anti-Fungal Antibodies

The EC50 value is a measure of the half-maximal effective concentration. The EC50 was calculated using laminarin binding ELISA, as detailed below.

ELISA plates were coated with 10 μg/mL Laminarin (Laminarin from Laminaria digitata, Sigma L9634) in PBS overnight at 5° C., then blocked with 3% BSA in PBST for 2-3 hours at room temperature. 3-fold serially diluted antibodies (2G8 variants) starting at 10 g/mL were applied onto the plates and incubated for 1 h at room temperature. The laminarin-bound antibodies were detected by HRP-conjugated rabbit anti-human IgG FC (Invitrogen, Rockford, IL) at a 1:20,000 dilution. The tetramethylbenzidine (TMB) substrate (SeraCare Life Sciences INC, Milford, MA, USA) was used for detection; absorbance at 450 nm was measured on a plate reader. Graphs were plotted (FIG. 1) and EC50's were calculated with GraphPad Prism 8.0 software (Table 5). Two KBio made antibodies (not 2G8) were used as negative controls and showed no binding to Laminarin.

TABLE 5

| Test Article | EC50 (μg/mL) |
|---|---|
| Clone 14.1 Reference | 0.05073 |
| Clone 3.1 | 0.05124 |
| Clone 14.1 | 0.037125 |
| Clone 32.3 | 1.5845 |
| Clone 39.1 | 0.05649 |
| Clone 40.1 | 0.017 |
| Clone 41.2 | 0.02878 |
| Clone 42.1 | 0.1356 |
| Clone 43.1 | 0.05045 |

### Example 2—Laminarin Binding (Biacore-SPR) to Determine the Binding Affinity (K_{D}) of Initial Humanized and Next Generation Affinity Matured Anti-Fungal Antibodies

The equilibrium binding constants (K_{D}) is a measure of the binding affinity of an antibody to its target, which may be determined by any suitable assay, such as surface plasmon resonance (SPR).

The binding affinity (K_{D}) of 2G8 variants to Laminarin was measured on a Biacore T200 instrument at ambient temperature. For each cycle, variants of 2G8 were captured on a Pro A sensor chip, following the manufacturer's instructions, to a surface density of about 1300-1500 RUs. Serial dilutions of Laminarin were made in running buffer (HBS-EP+, Cytiva) and injected at a flow rate of 30 μL/min for a contact time of 120 s and a dissociation time of 600s. Between sample injections, the system was washed with Protein A wash buffer. A reference flow cell was utilized to correct response contributions such as bulk shifts that occur equally in the sample and reference flow cells. A blank cycle (running buffer) was performed, and all sample injections were blank subtracted to correct the sensorgrams for drifts and other disturbances that affect the reference subtracted curve. A replicate of a non-zero concentration of Laminarin and the blank were injected in each experiment for double referencing, thus verifying the reliability of the Pro A chip throughout the experiment. The data were assessed by kinetics binding analysis for Association rate constant (ka) (FIG. 2B; Table 6), Dissociation rate constant (kd) (FIG. 2C; Table 7), and Equilibrium dissociation constant (K_{D}) (FIG. 2A; Table 8).

TABLE 6

| Test Article | Ka (1/Ms) |
|---|---|
| Clone 14.1 Reference | 121000 |
| Clone 3.1 | 165500 |
| Clone 14.1 | 125440 |
| Clone 32.3 | 73900 |
| Clone 39.1 | 98445 |
| Clone 40.1 | 135200 |
| Clone 41.2 | 97815 |
| Clone 42.1 | 138300 |
| Clone 43.1 | 92245 |

TABLE 7

| Test Article | Kd (1/s) |
|---|---|
| Clone 14.1 Reference | 0.0004435 |
| Clone 3.1 | 0.000471 |
| Clone 14.1 | 0.00045028 |
| Clone 32.3 | 0.000921 |
| Clone 39.1 | 0.0004966 |
| Clone 40.1 | 0.00024695 |
| Clone 41.2 | 0.0002927 |
| Clone 42.1 | 0.0006862 |
| Clone 43.1 | 0.00064185 |

TABLE 8

| Test Article | K_{D} (M) |
|---|---|
| Clone 14.1 Reference | 3.83e−009 |
| Clone 3.1 | 2.85e−009 |
| Clone 14.1 | 3.63e−009 |
| Clone 32.3 | 1.25e−008 |
| Clone 39.1 | 5.05e−009 |
| Clone 40.1 | 1.83e−009 |
| Clone 41.2 | 2.96e−009 |
| Clone 42.1 | 4.97e−009 |
| Clone 43.1 | 6.96e−009 |

### Example 3—Aspergillus fumigatus Challenge in Immunosuppressed Mice Followed by Treatment with the Initial Humanized Anti-Fungal Antibody or the Next-Generation, Humanized and Affinity Matured Antibody and Fungal Load Assessment

In the Murine model of invasive aspergillosis, animals were first subject to an immunosuppressive regime. All animals were administered cyclophosphamide (250 mg/kg) by intraperitoneal injection and cortisone acetate (500 mg/kg) by subcutaneous administration on Day-2 (relative to inoculation on Day 0). On Day 3 all animals were administered cyclophosphamide (250 mg/kg) by intraperitoneal injection and cortisone acetate (500 mg/kg) by subcutaneous administration. Bodyweights were recorded prior to dosing on Days −2 and 3 and animals were dosed with a volume of 10 ml/kg.

All test groups were given 15 mg/kg of mAb, which was formulated in 20 mM L-Histidine+240 mM Sucrose at pH 6.0, at 24 hours and 120 hours post-Aspergillus challenge. The vehicle control groups received formulation buffer (20 mM L-Histidine+240 mM Sucrose, pH 6.0). All groups were dosed with 2.5 mL/kg of test item (mAb or vehicle).

Immunosuppressed mice were monitored up to 14 days post infection with Apergillus fumigatus and the percentage survival was recorded each day.

As expected, mice that were challenged with A. fumigatus and provided only the control vehicle saw a 0% survival rate at day 8.

The mouse group that was given the vehicle control or clone 14.1 antibody showed 0% survival after 8 days. The mouse group that given clone 39.1 or 41.2 antibody, achieved a survival rate of 10%, and the mouse group that was given clone 3.1, 32.3, 40.1, 42.1, or 43.1 antibody achieved a survival rate of 20% (FIG. 3A).

The galactomannan levels in the BAL were analyzed by ELISA (Platelia Aspergillus Ag #62794, Bio-Rad) as per manufacturer's instructions.

Histopathological assessment was performed on lungs samples following completion of the BAL procedure. Briefly, the thorax of animals was opened and the lungs were excised and weighed. The whole lung was then insufflated with 10% neutral buffered formalin for a minimum of 24 hours and the tissues will be processed to paraffin blocks. Lung tissues approximately 4-5 μm sections were stained with haematoxylin and eosin (H&E) and Periodic Acid Schiff (PAS). Microscopic evaluation of the lungs included a grading of the inflammatory cell infiltrate and tissue inflammation (H&E) with particular consideration being made to the presence of fungal spores and hyphae (PAS).

The data show that the majority of animals from Groups 2-10, culled for welfare reasons prior to Day 14; had detectable levels of Fungi in the lungs by either BAL Galactomannan levels or PAS positive staining or both (FIG. 3B). There are only 2 animals (Animal 39 from Group 32.3 and Animal 47 from Group 39.1) where this is not the case.

None of the surviving animals in groups Saline/vehicle, 42.1 or 3.1 had detectable levels of Fungi in the lungs by either BAL Galactomannan levels or PAS positive staining. 1 survivor in group 32.3 had PAS positive staining while the other was negative in both, the lone survivor in group 39.1 had positive galactomannan BAL, 1 of two survivors in group 40.1 had positive galactomannan BAL while the other was negative in both, the lone survivor in group 41.2 had positive galactomannan BAL, 1 of two survivors in group 43.1 had positive PAS staining while the other did not have detectable levels in either assay.

### Example 4—Thermal Shift Assay (TSA) to Determine Antibody Melting Temperatures

A Thermal Shift Assay (TSA) was carried out to determine antibody melting temperatures. The melting temperatures (Tm) of antibodies were determined by the TSA performed on a Life Technologies qPCR system. Each antibody, at a final concentration of 0.5 mg/mL in Protein Thermal Shift™ Buffer (Applied Biosystems Cat. #4462263), was mixed with 1× Protein Thermal Shift™ Dye (Applied Biosystems Cat. #4462263) for a total volume of 20 μl/well in a MicroAmp™ Optical Reaction 96 well Plate. Blank controls were set up for protein alone. Samples were analyzed in triplicate. The plate was heated from 25° C. to 99° C. in 0.05° C./s increments. Data was analyzed using the Protein Thermal Shift software version 1.4 (Applied Biosystems Cat. #4466037). Melting temperatures (Tm) were plotted and displayed for Tm1 (FIG. 4A; Table 9) and Tm2 (FIG. 4B; Table 10).

TABLE 9

| Test Article | Tₘ₁(° C.) |
|---|---|
| Clone 3.1 | 66.196 |
| Clone 14.1 | 65.457 |
| Clone 32.3 | 65.457 |
| Clone 39.1 | 65.556 |
| Clone 40.1 | 64.866 |
| Clone 41.2 | 66.935 |
| Clone 42.1 | 65.014 |
| Clone 43.1 | 64.964 |

TABLE 10

| Test Article | Tₘ₂(° C.) |
|---|---|
| Clone 3.1 | 76.390 |
| Clone 14.1 | 82.644 |
| Clone 32.3 | 80.379 |
| Clone 39.1 | 81.265 |
| Clone 40.1 | 83.087 |
| Clone 41.2 | 77.917 |
| Clone 42.1 | 79.689 |
| Clone 43.1 | 80.231 |

1. An antibody, or antigen-binding fragment thereof, wherein the antibody or antigen-binding fragment comprises a light chain variable region comprising:
(a) a CDRL1 of SEQ ID NO 1;
(b) a CDRL2 selected from the group consisting of SEQ ID NO 2 and SEQ ID NO 3; and
(c) a CDRL3 of SEQ ID NO 4;
and a heavy chain variable region comprising:
(d) a CDRH1 selected from the group consisting of SEQ ID NOs 5-7;
(e) a CDRH2 selected from the group consisting of SEQ ID NOs 8-12 and SEQ ID NO 28;
and
(f) a CDRH3 of SEQ ID NO 13.

2. The antibody, or antigen-binding fragment thereof, of claim 1, wherein the light chain variable region comprises:
(a) a CDRL1 of SEQ ID NO 1;
(b) a CDRL2 of SEQ ID NO 2; and
(c) a CDRL3 of SEQ ID NO 4,
optionally wherein the light chain variable region comprises an amino acid sequence having at least 70% identity to SEQ ID NO 14.

3. The antibody, or antigen-binding fragment thereof, claim 1 or claim 2, wherein the heavy chain variable region comprises:
(a) a CDRH1 of SEQ ID NO 5;
(b) a CDRH2 of SEQ ID NO 8; and
(c) a CDRH3 of SEQ ID NO 13,
optionally wherein the heavy chain variable region comprises an amino acid sequence having at least 70% identity to SEQ ID NO 16.

4. The antibody, or antigen-binding fragment thereof, of any one of claim 1 or claim 2,
wherein the heavy chain variable region comprises:
(a) a CDRH1 of SEQ ID NO 5;
(b) a CDRH2 of SEQ ID NO 10; and
(c) a CDRH3 of SEQ ID NO 13,
optionally wherein the heavy chain variable region comprises an amino acid sequence having at least 70% identity to SEQ ID NO 18.

5. The antibody, or antigen-binding fragment thereof, of claim 1, wherein the light chain variable region comprises:
(a) a CDRL1 of SEQ ID NO 1;
(b) a CDRL2 of SEQ ID NO 3; and
(c) a CDRL3 of SEQ ID NO 4,
optionally wherein the light chain variable region comprises an amino acid sequence having at least 70% identity to SEQ ID NO 15.

6. The antibody, or antigen-binding fragment thereof, of any one of claim 1 2, or 5, wherein the heavy chain variable region comprises:
(a) a CDRH1 of SEQ ID NO 6;
(b) a CDRH2 of SEQ ID NO 9; and
(c) a CDRH3 of SEQ ID NO 13,
optionally wherein the heavy chain variable region comprises an amino acid sequence having at least 70% identity to SEQ ID NO 17.

7. The antibody, or antigen-binding fragment thereof, of claim 1 or claim 2 wherein the heavy chain variable region comprises:
(a) a CDRH1 of SEQ ID NO 5;
(b) a CDRH2 of SEQ ID NO 11; and
(c) a CDRH3 of SEQ ID NO 13,
optionally wherein the heavy chain variable region comprises an amino acid sequence having at least 70% identity to SED ID NO 19.

8. The antibody, or antigen-binding fragment thereof, of claim 1 or claim 2, wherein the heavy chain variable region comprises:
(a) a CDRH1 of SEQ ID NO 7;
(b) a CDRH2 of SEQ ID NO 12; and
(c) a CDRH3 of SEQ ID NO 13,
optionally wherein the heavy chain variable region comprises an amino acid sequence having at least 70% identity to SEQ ID NO 20.

9. The antibody, or antigen-binding fragment thereof, of claim 1 or claim 2, wherein the heavy chain variable region comprises:
(a) a CDRH1 of SEQ ID NO 6;
(b) a CDRH2 of SEQ ID NO 28; and
(c) a CDRH3 of SEQ ID NO 13,
optionally wherein the heavy chain variable region comprises an amino acid sequence having at least 70% identity to SEQ ID NO 29.

10. The antibody, or antigen-binding fragment thereof, of any one of claims 2 to 9 wherein the antibody, or antigen-binding fragment thereof, has at least 75%, at least 80%, at least 85%, at least 90%, at least 95%, at least 99%, or 100% sequence identity to the recited sequence.

11. The antibody, or antigen-binding fragment thereof, of any one of the preceding claims, wherein the antibody or antigen binding fragment is a single-chain variable fragment (scFV), a single-domain antibody (sdAb), an antigen-binding fragment (Fab), or a fragment antibody (F(ab′)₂).

12. The antibody, or antigen-binding fragment thereof, of any one of the preceding claims, wherein the antibody or antigen-binding fragment is IgG, IgA, IgM, IgE or IgD isotype or any allotype thereof.

13. The antibody, or antigen-binding fragment thereof, of any one of the preceding claims, wherein the antibody or antigen-binding fragment is humanized.

14. The antibody, or antigen-binding fragment thereof, of any one of the preceding claims, wherein the antibody binds to a β-glucan.

15. The antibody, or antigen-binding fragment thereof, of any one of the preceding claims, for use in a bispecific antigen-binding protein, antibody, or antigen-binding fragment thereof, that binds to two different antigens or epitopes.

16. The antibody, or antigen-binding fragment thereof, of any one of the preceding claims, for use in a multi-specific antigen-binding protein, antibody, or antigen-binding fragment thereof, that binds to two or more different antigens or epitopes.

17. The antibody, or antigen-binding fragment thereof, of any one of the preceding claims, wherein the antibody, or antigen-binding fragment thereof, is in the form of a homo-dimer or homo-multimer.

18. The antibody, or antigen-binding fragment thereof, of any one of the preceding claims, wherein the antibody, or antigen-binding fragment thereof, have an EC50 less than 0.2 μg/mL.

19. The antibody, or antigen-binding fragment thereof, of any one of the preceding claims, wherein the antibody or antigen-binding fragment has a Kp value lower than 1.5×10⁻⁸ M.

20. The antibody, or antigen-binding fragment thereof, of any one of the preceding claims, wherein the antibody or antigen-binding fragment is thermostable up to 80° C.

21. The antibody, or antigen-binding fragment thereof, of any one of the preceding claims for use as an anti-fungal agent.

22. The antibody, or antigen-binding fragment thereof, of any one of the preceding claims, for use in the treatment of fungal infection.

23. The antibody, or antigen-binding fragment thereof, of any one of the preceding claims, for use in the manufacture of a medicament for the treatment of fungal infection.

24. A method of treating fungal infection comprising administering the antibody, or antigen-binding fragment thereof, of any one of the preceding claims.

25. The antibody, or antigen-binding fragment thereof, for use according to claims 21-23 or the method of claim 24, wherein the fungal infection is an Aspergillus infection.

26. The antibody, or antigen-binding fragment thereof, for use according to claim 21-23 or the method of claim 24, wherein the fungal infection is a Candida infection.

27. A recombinant fusion protein wherein the fusion protein comprises an antibody, or antigen-binding fragment thereof, of any of the preceding claims, and a peptide toxin.

28. The recombinant fusion protein of claim 27 wherein:
(a) at least one of SEQ ID NO 14 and SEQ ID NO 16,
(b) at least one of SEQ ID NO 15 and SEQ ID NO 17,
(c) at least one of SEQ ID NO 14 and SEQ ID NO 18,
(d) at least one of SEQ ID NO 14 and SEQ ID NO 19,
(e) at least one of SEQ ID NO 14 and SEQ ID NO 20,
(f) at least one of SEQ ID NO 14 and SEQ ID NO 17, or
(g) at least one of SEQ ID NO 14 and SEQ ID NO 29 are linked to the peptide toxin.

29. The antibody, or antigen-binding fragment thereof, of any one of the preceding claims wherein the antibody, or antigen binding fragment thereof, is conjugated to a coupling moiety.

30. The antibody, or antigen-binding fragment thereof, of claim 29 wherein the coupling moiety is a drug, toxin, cytokine, enzyme or enzyme.

31. The antibody, or antigen-binding fragment thereof, of any one of claims 29-30, wherein the antibody, or antigen-binding fragment thereof, and the coupling moiety are conjugated by a linker molecule.

32. The antibody, or antigen-binding fragment thereof, of claim 31 wherein the linker molecule is a cleavable linker molecule.

33. A nucleic acid encoding an antibody, or an antigen-binding fragment thereof, according to any one of the preceding claims.
